(19) United States
(12) Patent Application Publication
(10) Pub. No.: US 2025/0241890 A1
(43) Pub. Date: Jul. 31, 2025

(54) 1,4-DIUREAS AND 1,4-DITHIOUREAS AND USES THEREOF

(71) Applicant: Purdue Research Foundation, West Lafayette, IN (US)

(72) Inventors: Jessica Sonia FORTIN, West Lafayette, IN (US); Susantha K. GANEGAMAGE, West Lafayette, IN (US)

(21) Appl. No.: 19/036,692

(22) Filed: Jan. 24, 2025

Related U.S. Application Data
(60) Provisional application No. 63/624,918, filed on Jan. 25, 2024.

(51) Int. Cl. A61K 31/404; A61K 31/17; A61P 25/28; C07C 275/28; C07D 403/12

(52) U.S. Cl. CPC A61K 31/404; A61K 31/17; A61P 25/28; C07C 275/28; C07D 403/12

(57) ABSTRACT

Compounds of the formulae (I), (II), (Ia), (Ib), and (Ic); pharmaceutical compositions comprising the compounds of formulae (I), (II), (Ia), (Ib), and (Ic) and a pharmaceutically acceptable carrier; and methods for using the compounds and pharmaceutical compositions in a method of inhibiting α-synuclein (α-syn) protein aggregation in a subject having, or at risk for, α-syn protein aggregation.

### CROSS-REFERENCE TO RELATED APPLICATIONS

This application claims the benefit of U.S. Provisional Application No. 63/624,918, filed Jan. 25, 2024, which is incorporated by reference as if fully set forth herein.

### STATEMENT OF GOVERNMENT SUPPORT

This invention was made with government support under AG071985 awarded by the National Institutes of Health. The government has certain rights in the invention.

### TECHNICAL FIELD

This disclosure relates to 1,4-diurea and 1,4-dithiourea substituted aromatic derivatives, compositions comprising same, and the use of such compounds and compositions to inhibit alpha-synuclein (α-syn) protein aggregation, including, but not limited to, oligomers and neurofibrillary tangles (NFTs), associated with synucleinopathies (e.g., Parkinson's disease (PD), dementia with Lewy bodies (DLB), and multiple system atrophy (MSA)).

### BACKGROUND

Parkinson's disease (PD) is a multifactorial chronic neurodegenerative disease that affects 10 million people worldwide and currently is reported to affect 90,000 people in the US alone. Important in the physiopathology of PD are Lewy bodies (LB), which are small inclusions found in the neurons located in the nuclei of the brainstem responsible for the control of fine motor movement (Shamoradian et al., 2019). These small inclusions are generated from misfolding and accumulation of alpha-synuclein (α-syn), a protein responsible for neuronal transmission, more precisely the release of α-syn into the synaptic cleft (Cardinale et al., 2021). Bodies of evidence have demonstrated the involvement of LB in neurodegeneration (Mahul-Mellier et al., 2020). The oligomer formation seems to be more toxic compared to the fibrils generated from misfolded α-syn (Winner et al., 2011). Many studies are focused on understanding the spreading of the misfolded α-syn, which seems to be a major event taking place in the physiopathology of PD. Currently, FDA-approved drugs are alleviating symptoms, and there are no small-molecule therapeutics available to delay the formation of LB.

Research efforts have focused on the discovery of small molecular scaffolds based on sulfonyls (Robustelli et al., 2022), sulfonamides (Fortin et al., 2022; and Kurnik et al., 2018), triazoles (Vittorio et al., 2020), thiadiazol (Ross et al. 2010), urea (Fortin et al., 2016; and Maity et al., 2022), amine (Ramirez et al., 2023; and Ramirez et al. 2023) or aminoindoles (Maity et al., 2022; and Ramirez et al., 2023), to impede the formation of oligomers. Urea is a biologically less toxic, stable hydrophilic functional moiety that is caused to convert and store nitrogenous waste as urea in the terrestrial animal body as a metabolic by-product until it is excreted (Adeyomoye et al., 2022). The biological activity and therapeutic effects of urea have not been fully investigated (Adeyomoye et al., 2022). However, some researchers have proven that urea and thiourea can interact with proteins to inhibit their aggregation (Fortin et al., 2016; Maity et al., 2022; Jahan et al., 2018; and Bhat et al., 2023) hey have specifically demonstrated a chaotropic effect via disintegrating inter- and intra-molecular non-covalent interactions (Rabilloud et al., 2009).

The anti-aggregation properties of aminoindole have been previously described, and aminoindole has been used as a building block to design a new family of anti-aggregation compounds, with a special focus on anti-oligomer properties (Ramirez et al., 2023). The effects of urea- and thiourea-based compounds, especially on α-syn oligomer and fibril formation, have not been explored.

In view of the above, a series of aminoindole-derived diurea and dithourea bioisosteric analogs was synthesized, and their effect on α-syn misfolding and aggregation was examined. It is an object of the present disclosure to provide new compounds and compositions and methods of using such compounds and compositions to inhibit α-syn oligomer and fibril formation. This and other objects and advantages, as well as inventive features, will be apparent from the detailed description provided herein.

### SUMMARY

Provided are compounds of formula I:

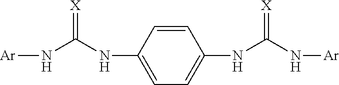

wherein X is O or S,
and a compound of formula II:

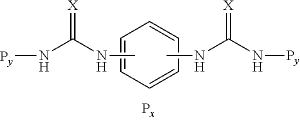

wherein Pₓ is:

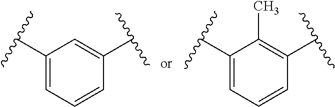

wherein X is O or S, and
each Ar is independently

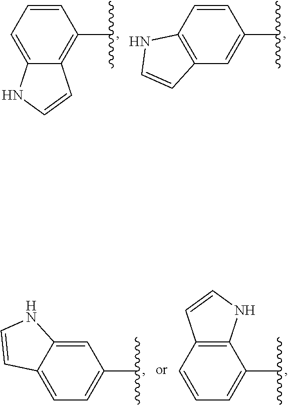

in which at least one ring carbon atom on each Ar is optionally independently substituted with C₁-C₆ alkyl or halo. Halo can be Cl, F, I or Br. In an embodiment of the compound of formula I, X is O and each Ar is

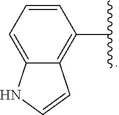 [SMILES: *c1cccc2[nH]ccc12]

In another embodiment of the compound of formula I, X is S and each Ar is

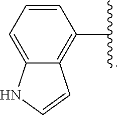 [SMILES: *c1cccc2[nH]ccc12]

in yet another embodiment or the compound of formula I, X is O and each Ar is

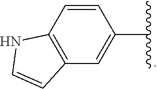 [SMILES: *c1ccc2[nH]ccc2c1]

In still yet another embodiment of the compound of formula I, X is S and each Ar is

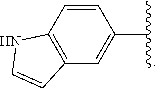 [SMILES: *c1ccc2[nH]ccc2c1]

In a further embodiment of the compound of formula I, X is O) and each Ar is

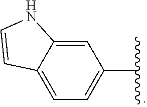 [SMILES: *c1ccc2cc[nH]c2c1]

In a yet further embodiment of the compound of formula I, X is S and each Ar is

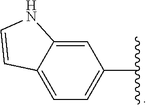 [SMILES: *c1ccc2cc[nH]c2c1]

In a still yet further embodiment of the compound of formula I, X is O and each Ar is

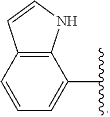 [SMILES: *c1cccc2cc[nH]c12]

In another embodiment of the compound of formula I, X is S and each Ar is

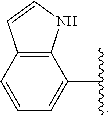 [SMILES: *c1cccc2cc[nH]c12]

Also provided is a pharmaceutical composition. The pharmaceutical composition comprises an above-described compound and a pharmaceutically acceptable carrier.

A method of inhibiting α-synuclein (α-syn) protein aggregation in a subject having, or at risk for, α-syn protein aggregation is also provided. The method comprises administering to the subject the above-described pharmaceutical composition in an amount effective to inhibit α-syn protein aggregation, whereupon α-syn protein aggregation is inhibited in the subject having, or at risk for, α-syn protein aggregation. The subject can have, or be at risk for, Alzheimer's disease, in which case the pharmaceutical composition can comprise the compound of formula I in which X is O and each Ar is

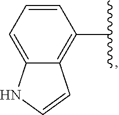 [SMILES: *c1cccc2[nH]ccc12]

X is S and each Ar is

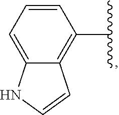 [SMILES: *c1cccc2[nH]ccc12]

or X is S and each Ar is

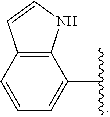 [SMILES: *c1cccc2cc[nH]c12]

The subject can have, or be at risk for, dementia with Lewy bodies (DLB). The subject can have, or be at risk for, multiple system atrophy (MSA). The subject can have neuroblastoma, and the formation of α-syn inclusions can be inhibited, such as with a pharmaceutical composition comprising the compound of formula in which X is O and each Ar is

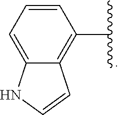 [SMILES: *c1cccc2[nH]ccc12]

### DESCRIPTION OF THE FIGURES

FIG. 1. Aminoindoles inhibited α-synuclein (α-syn) oligomer formation by PICUP. α-Syn (60 μM) was cross-linked (PICUP assay) with dimethylsulfoxide (DMSO; control) or compound at 50 μM (˜molar ratio, 1:1). 4-, 5-, 6-aminoindoles, but not the 2-(4-aminophenyl)benzothiazole (Ramirez et al. 2023), abrogated the formation of high molecular weight bands (i.e., oligomers) visible between 35 and 40 KDa. Coomassie blue-stained polyacrylamide gels showed high molecular weight α-syn oligomers with control (0.125% DMSO). Additional controls consisted of no light and no cross-linking agent (no Ru(bpy)), which provided no cross-linked products.

FIG. 2. Docking of analog 2 (green color on left) and 1 (yellow color on right) and their binding locations on human α-syn (PDB ID: 1XQ8) within the active site (middle) with hydrogen bonding.

FIG. 3. ThT kinetic curves (A) and dose-dependent reduction of α-synuclein (α-syn) fibrillation by compound 1 (B) and compound 2 (C). A) Kinetic curves were achieved using 100 μM of compounds 1 and 2. The control consisted of the vehicle (DMSO at 0.25%). B and C) The dose-dependent curves resulted from incubations carried out with α-syn at 47 hours with four concentrations (i.e. 12.5, 25, 50, and 100 μM) of the best α-syn anti-fibrillary compounds 1 and 2 of the diurea/dithiourea analogs. Triplicate data were collected from ten consecutive time points at the plateau phase. For both experiments, α-syn was tested at 6 μM.

FIG. 4. Compounds 1, 2, and 8 reduced α-syn oligomer formation by PICUP. α-Syn (60 μM) was cross-linked (PICUP assay) with DMSO (control) or compound at 50 μM (˜molar ratio, 1:1). Compounds 3, 4, 5, 6 reduced slightly the formation of high molecular bands (i.e., oligomers). Pixel density of the high molecular weight bands labeled as oligomers and the low molecular bands identified as monomers were quantified by image J. The relative pixel density (RPD) was obtained from dividing the pixel density of the higher molecular bands for each condition by their respective monomeric band.

FIG. 5. 1,4-Diurea and dithiourea substituted aromatic representatives failed to inhibit tau 0N4R oligomer formation by PICUP. Compounds 1, 2, and 8 were selected for their α-syn anti-oligomerization activity. Compound 7 was used as di-urea counterpart of compound 8, as compound 1 is the di-urea counterpart of compound 2, a dithiourea derivative. Tau (10 μM) was cross-linked (PICUP assay) with different concentrations of the di-substituted aminoindolyl derivatives at 50 μM (molar ratio 1:5).

FIG. 6. 1,4-Diurea and dithiourea substituted aromatic representatives did not prevent p-tau 1N4R oligomer formation by PICUP. p-Tau isoform 1N4R (4 μM) was cross-linked (PICUP assay) with 50 μM of compounds 1 and 2 (molar ratio 1:12.5).

FIG. 7. Compounds 1 and 2 treatments resulted in less and shorter α-syn fibrils by transmission electron microscopy (TEM). α-Syn (2 μM) was incubated with DMSO (0.25%; ‘CTRL’) or compound 1 or 2 (at 100 μM) for ˜22 hours prior to TEM visualization. Scale bars=200 nm.

FIG. 8. Compounds 1 and 2 are not toxic to neuroblastoma (SH-SY5Y) cells at low micromolar concentration. The evaluation of cell density was conducted using crystal violet staining. Statistical analysis utilizing a one-way analysis of variance (ANOVA) with Dunnett's multiple comparison test indicated no significant differences in percentage of cells between DMSO-treated cells and those subjected to different pharmacologic treatments.

FIG. 9. Compounds 1 and 2 do not exhibit any cytotoxicity to neuroblastoma (SH-SY5Y). Cells were treated for 48 hours with 20 μM of compounds 1 or 2 or 0.05% DMSO. Cell survival was measured using MTT method. Two replicates per condition were averaged together.

FIG. 10. Compounds 1 and 2 do not exhibit any cytotoxicity to neuroblastoma (SH-SY5Y). Cells were treated for 48 hours with A) 5 μM and B) 10 μM of compounds 1 or 2 or DMSO. Cell survival was measured using MTT method. 4 replicates per condition were averaged together.

FIG. 11. Compound 1 prevents αS inclusion formation. M17D cells expressing the inclusion-prone αS-3K::YFP fusion protein (dox-inducible) were treated with 0.1% DMSO (vehicle; “0 μM”) as well as 1.25, 2.5, 5 and 10 μM of compounds 1, 2 and 8 at t=24 hours after plating. Cells were induced with doxycycline at t=48 hours. A) Incucyte-based analysis of punctate YFP signals relative to 0.1% DMSO was done at t=96 hours (N=3 independent experiments, n=6-18 individual wells total (0 μM, n=18; 10 μM, n=6; all other concentrations, n=12). B) Same as panel A, but confluence fold changes relative to DMSO vehicle (0 μM) were plotted. C) Representative IncuCyte images of reporter cells treated with vehicle vs. 10 μM compounds 1, 2, or 8 (t=96 h), green channel. Arrows indicate αS-rich YFP-positive inclusions. Scale bar, 50 μm. All data are presented as fold-changes relative to DMSO control +/−standard deviation. One-way ANOVA, Dunnett's post-hoc test; *, p<0.05; **, p<0.01; ***, p<0.001.

### DESCRIPTION

The present disclosure is based on the evaluation of ten di-urea/di-thiourea compounds with a 1,4-phenyl diurea/thiourea linkage for anti-aggregation properties against α-synuclein (α-syn) aggregation. To characterize such anti-fibrillary activity, thioflavin T (ThT) fluorescence assays and transmission electron microscopy (TEM) were utilized. The anti-oligomer activity of the best compounds was assessed with several prone-to-aggregate proteins by photo-induced cross-linking of unmodified protein (PICUP) assay. Afterward, the biological effects of the best compounds were studied using the M17D intracellular inclusion cell-based model. Based on the anti-fibrillary biophysical assays, compounds 1 and 2 (a 1,4-phenyl urea or thiourea with two 4-aminoindolyls) were identified as the most promising lead molecules in the series of compounds. Compounds 2 and 8, and to a lesser extent compound 1, were effective in reducing α-syn oligomer formation but not tau isoform 0N4R and p-tau isoform 1N4R. Interestingly, the cell-based studies showed no α-syn anti-inclusion effect of compound 8, which exhibited a modest α-syn anti-fibrillar and greatest α-syn anti-oligomer effects. A significant reduction in M17D α-syn inclusion was detected following compound 1 treatment at low micromolar concentration, but resulted in the opposite effect, i.e., an increase in α-syn inclusion, in cells treated with compound 2 using the same concentration (10 μM). Importantly, no changes in confluence with compounds 1, 2, and 8 were noticed. These results demonstrate that compound 2 failed to prevent α-syn inclusion in a cell-based model, while compound 1 was effective in reducing inclusion, and neither of the two compounds display cytotoxic effects. These results are indicative that the anti-oligomer activity of a compound doesn't not necessarily correlate with anti-inclusion activity. This study was performed to provide insight into anti-fibrillar, anti-oligomer, anti-inclusion effects of symmetric molecules specific to 1,4-diurea-phenyl and 1,4-dithiourea-phenyl substituted with aminoindolyl, with the 4-aminoindolyl resulting in the best biophysical and biological activities with the assays performed.

In view of the above, provided is a compound of formula I:

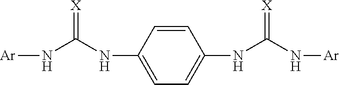

wherein X is O or S,
and a compound of formula II:

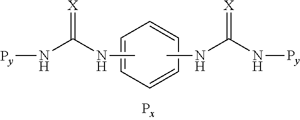

wherein Pₓ is:

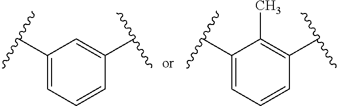

wherein X is O or S, and
each Ar is independently

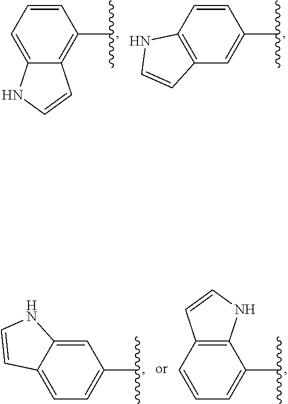

in which at least one ring carbon atom on each Ar can be optionally independently substituted with C₁-C₆ alkyl or halo. C₁-C₆ alkyl refers to a fully saturated cyclic or acyclic, branched or unbranched carbon chain moiety having the number of carbon atoms specified. For example, an alkyl of 1 to 6 carbon atoms refers to moieties such as methyl, ethyl, propyl, butyl, pentyl, and hexyl. Alkyl groups may be unsubstituted or substituted. Halo can be Cl, F, I or Br. In an embodiment of the compound of formula I, X is O and each Ar is

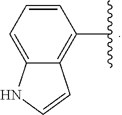 [SMILES: *c1cccc2[nH]ccc12]

In another embodiment of the compound of formula I, X is S and each Ar is

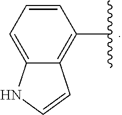 [SMILES: *c1cccc2[nH]ccc12]

In yet another embodiment of the compound of formula I, X is O and each Ar is

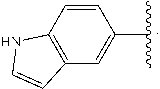 [SMILES: *c1ccc2[nH]ccc2c1]

In still yet another embodiment of the compound of formula I, X is S and each Ar is

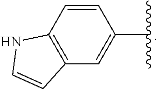 [SMILES: *c1ccc2[nH]ccc2c1]

In a further embodiment of the compound of formula I, X is O and each Ar is

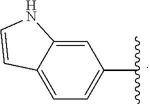 [SMILES: *c1ccc2cc[nH]c2c1]

In a yet further embodiment of the compound of formula I, X is S and each Ar is

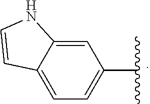 [SMILES: *c1ccc2cc[nH]c2c1]

In a still yet further embodiment of the compound of formula I, X is O and each Ar is

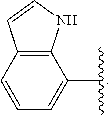 [SMILES: *c1cccc2cc[nH]c12]

In another embodiment of the compound of formula I, X is S and each Ar is

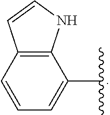 [SMILES: *c1cccc2cc[nH]c12]

Also provided are compounds of the formula:

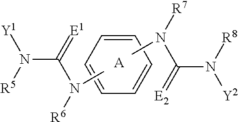

or a pharmaceutically acceptable salt thereof,
where the ring A is substituted by groups R¹-R⁴,
such as compounds of the formulae (Ia) and (Ib):

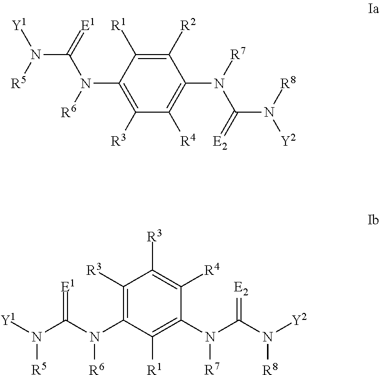

or a pharmaceutically acceptable salt thereof,
wherein:
E¹ and E² are each independently O, S, or NH;
R¹-R⁴ are each independently H, halogen, C₁-C₂₀ alkyl, or C₁-C₂₀ alkoxy;
R⁵-R⁸ are each independently H or C₁-C₂₀ alkyl;
Y¹ and Y² are each independently C₃-C₈ cycloalkyl; or

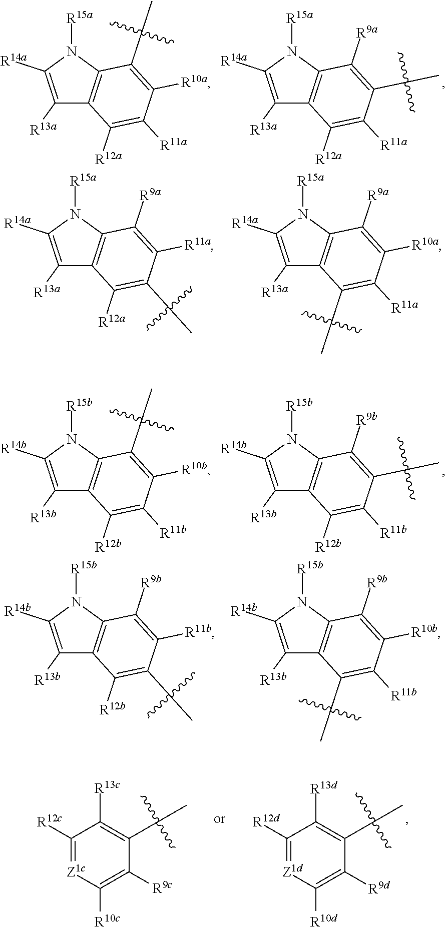

wherein
z^{1c} is N or C—R^{11c};
Z^{1d} is N or C—R^{11d}
R^{9c}-R^{15a}, R^{9b}-R^{15b}, R^{9c}-R^{15c}, and R^{9d}-R^{15d} are each independently H, halogen, —OH, —NH₂, —NO₂, —CF₃, —CN, —COOH, —N₃, —SO₃H, or —PO₃H₂, C₁-C₂₀ alkyl, C₁-C₂₀ alkoxy, C₂-C₂₀ alkenyl, C₂-C₂₀ alkynyl, C₃-C₁₂ cycloalkyl, C₃-C₁₂ cycloalkenyl, or 3- to 15-membered heterocyclyl group. In one example, R^{9a}-R^{15a}, R^{9b}-R^{15b}, R^{9c}-R^{15c}, and R^{9d}-R^{15d} can each be optionally substituted with one or more halogen, —OH, —NH₂, —NO₂, —CN, —COOH, —N₃, —SO₃H, or —PO₃H₂. In one example, R^{11c} and R^{11d} can each independently be a 6-membered heterocycle.

Also provided are compounds of the formula:

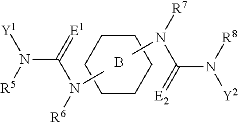

or a pharmaceutically acceptable salt thereof,
where the ring B is substituted by groups R¹-R⁴,
wherein:
R¹-R⁴ are each independently H, halogen, C₁-C₂₀ alkyl, or C₁-C₂₀ alkoxy;
such as a compound of the formula (Ic):

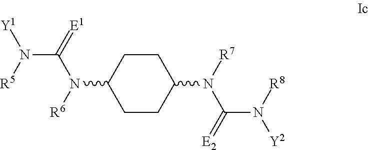

or a pharmaceutically acceptable salt thereof,
wherein:
E¹ and E² are each independently O, S, or NH;
R⁵-R⁸ are each independently H or C₁-C₂₀ alkyl;
Y¹ and Y² are each independently:

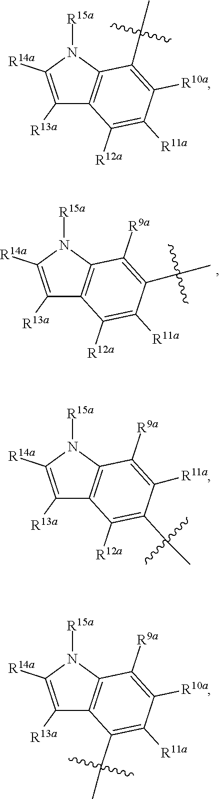

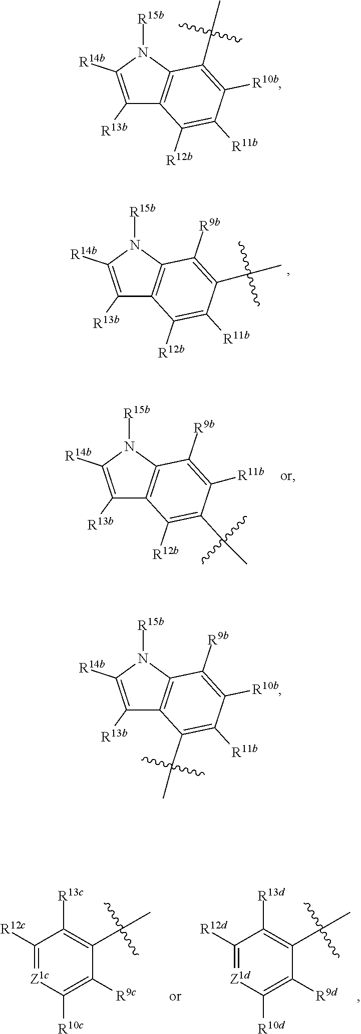

wherein
Z^{1c} is N or C—R^{11c};
Z^{1d} is N or C—R^{11d},
R^{9a}-R^{15a}, R^{9b}-R^{15b}, R^{9c}-R^{15c}, and R^{9d}-R^{15d} are each independently H, halogen, —OH, —NH₂, —NO₂, —CF₃, —CN, —COOH, —N₃, —SOSH, or —POSH₂, C₁-C₂₀ alkyl, C₁-C₂₀ alkoxy, C₂-C₂₀ alkenyl, C₂-C₂₀ alkynyl, C₃-C₁₂ cycloalkyl, C₃-C₁₂ cycloalkenyl, or 3- to 15-membered heterocyclyl group. In one example, R^{9a}-R^{15a}, R^{9b}-R^{15b}, R^{9c}-R^{15c}, and R^{9d}-R^{15d} can each be optionally substituted with one or more halogen, —OH, —NH₂, —NO₂, —CN, —COOH, —N₃, —SO₃H, or —PO₃H₂. In one example, R^{11c} and R^{11d} can each independently be a 6-membered heterocycle.

The compound of the formula (Ic) can be of the formula:

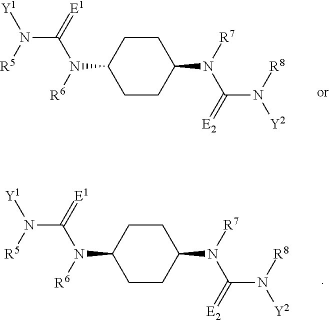

In any of the foregoing compounds (e.g., the compounds of the formulae (Ia), (Ib), and (Ic)), E¹ and E² can both be O. Alternatively, E¹ and E² can both be S. Alternatively, or in addition, in any of the foregoing compounds R¹-R⁴ can each H be; or R¹ is alkyl and R²-R⁴ can each be H. Alternatively, or in addition, in any of the foregoing compounds R⁵ and R⁶ can each independently be H. Alternatively, or in addition, in any of the foregoing compounds R⁷ and R⁸ can each independently H be. For example, in any of the foregoing compounds R⁵-R⁸ can each be H. In another example, in any of the foregoing compounds R¹ can be methyl. Alternatively, or in addition, in any of the foregoing compounds Y¹ and Y² can each independently be

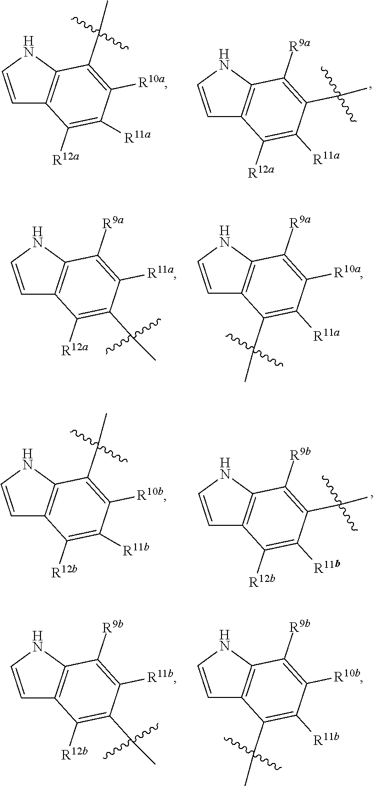

wherein R^{9a}-R^{15a} and R^{9b}-R^{15b} are each independently H, halogen or C₁-C₆ alkyl;
or a pharmaceutically acceptable salt thereof.

In any of the foregoing compounds (e.g., the compounds of the formulae (Ia), (Ib), and (Ic)), both Y¹ and Y² can each be

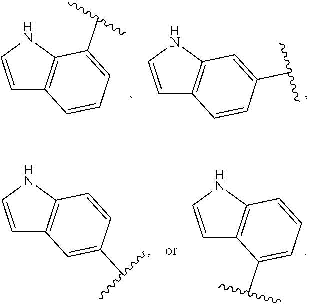

In another example, in any of the foregoing compounds (e.g., the compounds of the formulae (Ia), (Ib), and (Ic)), Y¹ and Y² can each independently be:

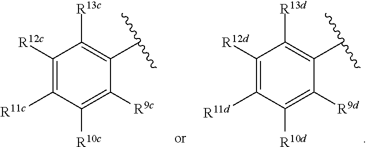

In one example, R^{11c} and R^{11d} can each independently be a 6-membered heterocycle, such as where Y¹ and Y² are each independently:

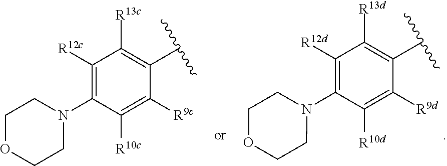

In yet another example, in any of the foregoing compounds (e.g., the compounds of the formulae (Ia), (Ib), and (Ic)), Y¹ and Y² can each independently be:

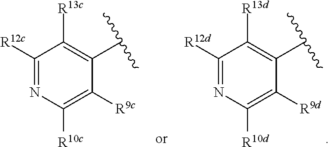

The disclosure also relates to a compound of the formula:

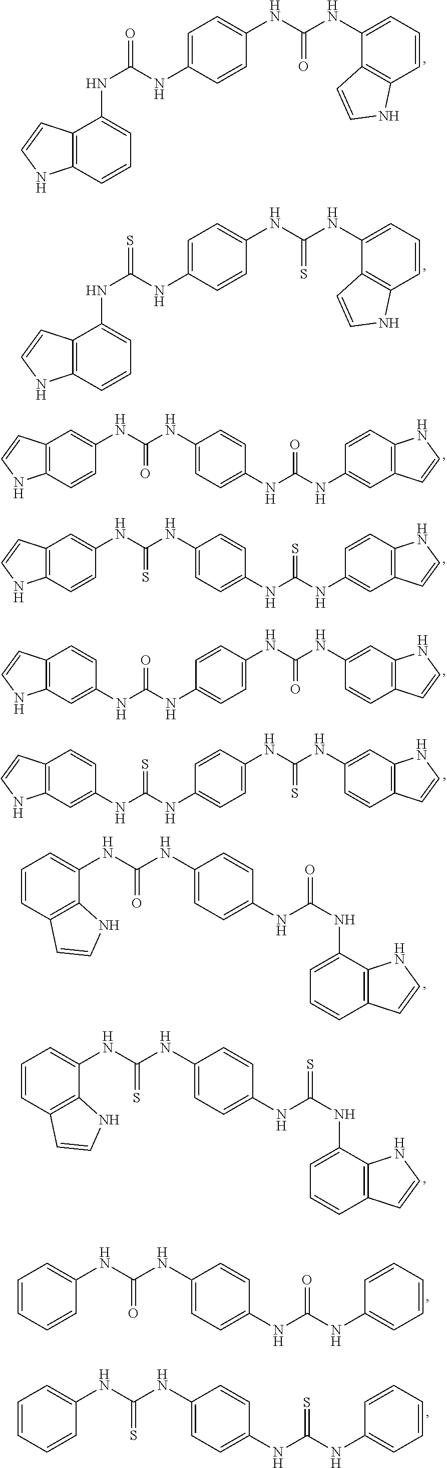

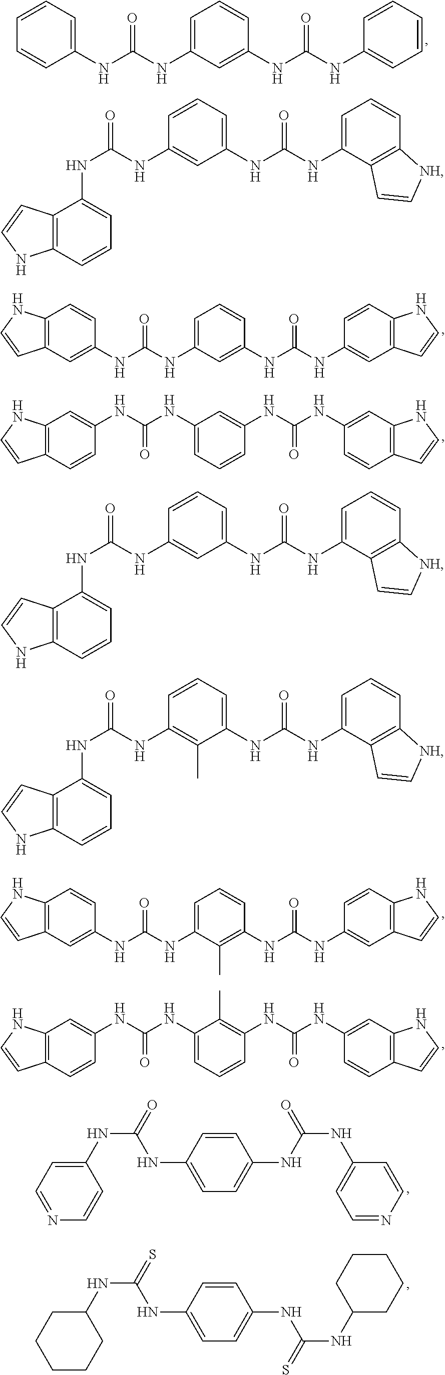

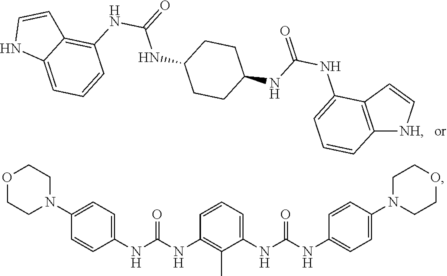 [SMILES: O=C(Nc1cccc2[nH]ccc12)N[C@H]1CC[C@H](NC(=O)Nc2cccc3[nH]ccc23)CC1 ; "or" ; Cc1c(NC(=O)Nc2ccc(N3CCOCC3)cc2)cccc1NC(=O)Nc1ccc(N2CCOCC2)cc1]

or a pharmaceutically acceptable salt thereof.

The above compounds include isotopic variants and compounds in which one or more hydrogen atoms have been substituted with deuterium. The compounds may contain one or more chiral centers or may otherwise be capable of existing as multiple stereoisomers. In one embodiment, the compounds are not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds may include geometric centers, such as cis, trans isomers, diastereomers, enantiomers, and E and Z double bonds. In another embodiment, the compounds are not limited to any particular geometric isomer requirement, and that the compounds, and compositions, methods, uses, and medicaments that include them may be pure, or may be any of a variety of geometric isomer mixtures. Such mixtures of geometric isomers may include a single configuration at one or more double bonds and chiral carbons, while including mixtures of geometry at one or more other double bonds and chiral carbons.

The terms “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference for its teachings regarding same.

The term “solvate” means a compound, or a salt thereof, that further includes a stoichiometric or non-stoichiometric amount of solvent bound by non-covalent intermolecular forces. Where the solvent is water, the solvate is a hydrate.

The above compounds, and pharmaceutically acceptable salts and solvates thereof, can be synthesized in accordance with methods known in the art and exemplified herein. See, e.g., Example 1.

The compounds can be used to inhibit the aggregation of proteins prone to aggregate in a state of disease. Proteins prone to aggregate include, but are not limited to, islet amyloid polypeptide, amyloid-β, α-synuclein (α-syn), tubulin associated unit (tau), and transthyretin. The tau can be tau isoform 2N₄R or 1N4R. The compounds described herein can be used to inhibit the aggregation of α-syn. Diseases involving protein aggregation include, but are not limited to, AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis. The compounds described herein can be used to inhibit the aggregation of α-syn in a subject having, or at risk for, Alzheimer's disease, dementia with Lewy bodies (DLB), or multiple system atrophy (MSA). The compounds described herein also can be used to inhibit the formation of α-syn inclusions in a subject with a neuroblastoma.

The compounds can be formulated as pharmaceutical compositions comprising a pharmaceutically acceptable carrier using methods well-known in the art. “Carrier” is used generically herein to refer to pharmaceutically acceptable carriers, diluents, adjuvants, and excipients. See, e.g., Remington. The Science and Practice of Pharmacy, 23^{rd} edition, Oct. 30, 2020, Adeboye Adejare, ed. Accordingly, further provided is a pharmaceutical composition comprising the compound of formula I and a pharmaceutically acceptable carrier.

Still further provided is a method of inhibiting α-synuclein (α-syn) protein aggregation in a subject having, or at risk for, α-syn protein aggregation. The method comprises administering to the subject the above-described pharmaceutical composition in an amount effective to inhibit α-syn protein aggregation, whereupon α-syn protein aggregation is inhibited in the subject having, or at risk for, α-syn protein aggregation. The subject can have, or be at risk for, Alzheimer's disease, in which case the pharmaceutical composition can comprise the compound of formula I in which X is O and each Ar is

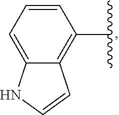 [SMILES: *c1cccc2[nH]ccc12]

X is S and each Ar is

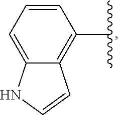 [SMILES: *c1cccc2[nH]ccc12]

or X is S and each Ar is

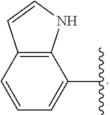 [SMILES: *c1cccc2cc[nH]c12]

The subject can have, or be at risk for, dementia with Lewy bodies (DLB). The subject can have, or be at risk for, multiple system atrophy (MSA). The subject can have neuroblastoma, and the formation of α-syn inclusions can be inhibited, such as with a pharmaceutical composition comprising the compound of formula in which X is O and each Ar is

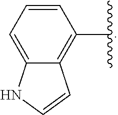 [SMILES: *c1cccc2[nH]ccc12]

The terms “substituted,” “substituent,” and “functional group” refer to the replacement of one or more hydrogen atoms on a molecule or group (e.g., on an aryl or an alkyl group) with another atom or group. Examples of substituents include, but are not limited to, a halogen (e.g., F, Cl, Br, and I), —OR, —OC(O)N(R)(R′), —CN, —NO, —NO₂, —ONO₂, —N₃, —CF₃, —OCF₃, —R, ═O (oxo), ═S (thiono), —C(O), —S(O), methylenedioxy, ethylenedioxy, —N(R)(R′), —SR, —SOR, —SO₂R, —SO₂N(R)(R′), —SO₃R, —(CH₂)₀₋₂P(O)(OR)(OR′), —C(O)R, —C(O)C(O)R, —C(O)CH₂C(O)R, —C(S)R, —C(O)OR, —OC(O)R, —C(O)N(R)(R′), —OC(O)N(R)(R′), —C(S)N(R)(R′), —(CH₂)₀₋₂N(R)C(O)R′, —(CH₂)₀₋₂N(R)C(O)OR′, —(CH₂)₀₋₂N(R)N(R′)(R″), —N(R)N(R′)C(O)R″, —N(R)N(R′) C(O)OR″, —N(R)N(R′)CON(R″)(R′″), —N(R)SO₂R′, —N(R)SO₂N(R′)(R″), —N(R)C(O)OR′, —N(R)C(O)R′, N(R)C(S)R′, —N(R)C(O)N(R′)(R″), —N(R)C(S)N(R′)(R″), —N(COR)COR′, —N(OR)R′, —C(═NH)N(R)(R′), —C(O)N(OR)R′, or —C(═NOR)R′, wherein R, R′, R″, and R′″ are each independently selected from hydrogen, alkyl, acyl, cycloalkyl, aryl, aralkyl, heterocyclyl, heteroaryl, or heteroarylalkyl, and wherein R and R′ when bonded to a nitrogen atom or to adjacent nitrogen atoms can together form a heterocyclyl, which can be mono- or multi-substituted.

The term “alkyl” as used herein refers to substituted or unsubstituted straight chain and branched mono- or divalent alkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), and 1 to 6 carbon atoms (C₁-C₆). The enumerated ranges also include all subranges, for example, C₁ to C₈ which includes C₁-C₂, C₁-C₃, C₁-C₄, C₁-C₅, C₁-C₆, C₁-C₇, C₁-C₈, C₂-C₃, C₂-C₄, C₂-C₅, C₂- C₆, C₂-C₇, C₂-C₈, C₃-C₄, C₃-C₅, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈, and C₇-C₈. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term ‘alkyl’ encompasses n-alkyl, isoalkyl, and ante-isoalkyl groups as well as other branched chain forms of alkyl. Representative substituted alkyl groups can be substituted one or more times with substituents selected from amino, hydroxy, cyano, carboxy, nitro, thio, alkoxy, and halogen groups.

The term “alkenyl” as used herein refers to substituted or unsubstituted straight chain and branched mono- or divalent alkenyl groups having at least one double bond and having from 2 to 40 carbon atoms (C₂-C₄₀), 2 to about 20 carbon atoms (C₂-C₂₀), 2 to 12 carbons (C₂-C₁₂), 2 to 8 carbon atoms (C₂-C₈), and 2 to 6 carbon atoms (C₂-C₆). The enumerated ranges also include all subranges, for example, C₂ to C₈ which includes C₂-C₃, C₂-C₄, C₂-C₅, C₂-C₆, C₂-C₇, C₂-C₈, C₃-C₄, C₃-C₈, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈, and C₇-C₈. Examples of straight chain alkenyl groups include those with from 2 to 8 carbon atoms such as —CH═CH—, —CH—CHCH₃, and —CH₂CH—CHCH₂-groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C═CH(CH₃) groups. Representative substituted alkenyl groups can be substituted one or more times with substituents selected from amino, hydroxy, cyano, carboxy, nitro, thio, alkoxy, and halogen groups.

The term “alkynyl” as used herein refers to substituted or unsubstituted straight chain and branched mono- or divalent alkynyl groups having at least one triple bond and having from 2 to 40 carbon atoms (C₂-C₄₀), 2 to about 20 carbon atoms (C₂-C₂₀), 2 to 12 carbons (C₂-C₁₂), 2 to 8 carbon atoms (C₂-C₈), and 2 to 6 carbon atoms (C₂-C₆). The enumerated ranges also include all subranges, for example, C₂ to C₈ which includes C₂-C₃, C₂-C₄, C₂-C₅, C₂-C₆, C₂-C₇, C₂-C₈, C₃-C₄, C₃-C₅, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈. Examples of straight chain alkynyl groups include those with from 2 to 8 carbon atoms such as —C≡CH, —C≡CCH₃, and —CH₂C≡CCH₂-groups. Examples of branched alkynyl groups include, but are not limited to, —C═C—CH—(CH₃)₂ and —CH₂C≡C—CH₃ groups. Representative substituted alkynyl groups can be substituted one or more times with substituents selected from amino, cyano, carboxy, alkoxy, and halogen groups.

The term “alkoxy” as used herein refers to —O-alkyl groups wherein the alkyl portion is as defined above and includes groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), and 1 to 6 carbon atoms (C₁-C₆). The enumerated ranges also include all subranges, for example, C₁ to C₈ which includes C₁-C₂, C₁-C₃, C₁-C₄, C₁-C₈, C₁-C₆, C₁-C₇, C₁-C₈, C₂-C₃, C₂-C₄, C₂-C₅, C₂-C₆, C₂-C₇, C₂-C₈, C₃-C₄, C₃-C₈, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈, and C₇-C₈. Examples include methoxy (—OCH₃), ethoxy (—OCH₂CH₃), propoxy (—OCH₂CH₂CH₃), and butoxy (—OCH₂CH₂CH₂CH₃) groups, as well as their branched isomers such as isopropoxy (—OCH(CH₃)₂ and tert-butoxy (O—C(CH₃)₃ groups. Representative substituted alkoxy groups can be substituted one or more times with substituents selected from amino, hydroxy, cyano, carboxy, nitro, thio, alkoxy, and halogen groups.

The terms “halo,” “halogen,” and “halide” group, by themselves or as part of another substituent, mean, unless otherwise stated, a fluorine, chlorine, bromine, or iodine atom (e.g. F, Cl, Br, or I).

The term “haloalkyl” as used herein refers to alkyl groups as defined above wherein one or more hydrogen atoms are replaced by halogen atoms (F, Cl, Br, or I). The haloalkyl groups can have from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), and 1 to 6 carbon atoms (C₁-C₆). 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), and 1 to 6 carbon atoms (C₁-C₆). The enumerated ranges also include all subranges, for example, C₁ to C₈ which includes C₁-C₂, C₁-C₃, C₁-C₄, C₁-C₅, C₁-C₆, C₁-C₇, C₁-C₈, C₂-C₃, C₂-C₄, C₂-C₅, C₂-C₆, C₂-C₇, C₂-C₈, C₃-C₄, C₃-C₈, C₃-C₆, C₃-C₇, C₃-C₈, C₄-C₅, C₄-C₆, C₄-C₇, C₄-C₅, C₅-C₆, C₅-C₇, C₅-C₈, C₆-C₇, C₆-C₈, and C₇-C₈. Examples include fluoromethyl (CH₂F), difluoromethyl (CHF₂), trifluoromethyl (CF₃), chloromethyl (—CH₂Cl), dichloromethyl (—CHCl₂), trichloromethyl (—CCl₃), 2-fluoroethyl (CH₂CH₂F), 2,2-difluoroethyl (—CH₂CF₂H), 2,2,2-trifluoroethyl (—CH₂CF₃), 2-chloroethyl (—CH₂CH₂Cl), 2,2-dichloroethyl (—CH₂CCl₂H), and 2,2,2-trichloroethyl (—CH₂CCl) As used herein, the term “haloalkyl” includes mono-halo alkyl groups and poly-halo alkyl groups, wherein all the halogen atoms can be the same or different, and per-halo alkyl groups, wherein all hydrogen atoms are replaced by halogen atoms, such as fluoro. Further examples of haloalkyls include 1,1-dichloroethyl, 1,2-dichloroethyl, 1,3-dibromo-3,3-difluoropropyl, perfluorobutyl, —CF(CH₃) 2 and the like.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₈). The enumerated ranges also include all subranges, for example, C₂ to C₈ which includes C₂-C₄, C₂-C₅, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈, and C₇-C₈. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “alkylcycloalkyl” as used herein refers to substituted or unsubstituted alkyl groups as defined herein in which a hydrogen or carbon bond of an alkyl group as defined herein is replaced with a bond to a cycloalkyl group as defined herein. The alkylcycloalkyl groups can have from 4 to 40 total carbon atoms (C₄-C₄₀), 4 to about 20 carbon atoms (C₄-C₂₀), 4 to 10 carbons (C₄-C₁₀), and 4 to 8 carbon atoms (C₄-C₈). The enumerated ranges also include all subranges, for example, C₄ to C₁₀, which includes C₄-C₅, C₄-C₆, C₄-C₇, C₄-C₈, C₅-C₆, C₅-C₇, C₅-C₅, C₆-C₇, C₆-C₈, and C₇-C₈, C₇-C₉, C₇-C₁₀, C₈-C₉, C₈-C₁₀, and C₉-C₁₀. The alkyl portion can have from 1 to 6 carbon atoms (C₁-C₆), and the cycloalkyl portion can have for example from 3 to 8 carbon atoms (C₃-C₈). Representative alkylcycloalkyl groups include, but are not limited to, methylcyclopropyl, methylcyclobutyl, methylcyclopentyl, methylcyclohexyl, ethylcyclopropyl, ethylcyclobutyl, ethylcyclopentyl, ethylcyclohexyl, and their higher homologs. Representative substituted alkylcycloalkyl groups can be substituted one or more times with substituents selected from amino, hydroxy, cyano, carboxy, nitro, thio, alkoxy, and halogen groups.

The term “cycloalkylalkyl” as used herein refers to substituted or unsubstituted cycloalkyl groups as defined herein in which a hydrogen of a cycloalkyl group as defined herein is replaced with a bond to an alkyl group as defined herein. The cycloalkylalkyl groups can have from 4 to 40 total carbon atoms (C₄-C₄₀), 4 to about 20 carbon atoms (C₄-C₂₀), 4 to 10 carbons (C₄-C₁₀), and 4 to 8 carbon atoms (C₄-C₈). The enumerated ranges also include all subranges, for example, C₄ to C₁₀, which includes C₄-C₅, C₄-C₆, C₄-C₇, C₄-C₈, C₅-C₆, C₅-C₇, C₅-C₈, C₆-C₇, C₆-C₈, and C₇-C₈, C₇-C₉, C₇-C₁₀, C₈-C₉, C₅-C₁₀, and C₉-C₁₀. The alkyl portion can have for example from 1 to 6 carbon atoms (C₁-C₆), and the cycloalkyl portion can have for example from 3 to 8 carbon atoms (C₃-C₈). Representative alkylcycloalkyl groups include, but are not limited to, methylcyclopropyl, methylcyclobutyl, methylcyclopentyl, methylcyclohexyl, ethylcyclopropyl, ethylcyclobutyl, ethylcyclopentyl, ethylcyclohexyl, and their higher homologs. Representative substituted alkylcycloalkyl groups can be substituted one or more times with substituents selected from amino, hydroxy, cyano, carboxy, nitro, thio, alkoxy, and halogen groups.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. “Aryl” and the phrase “aryl group” includes fused ring species including those that include fused aromatic and non-aromatic groups. Accordingly, “aryl” and the phrase “aryl group” include groups of the formula:

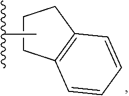

each of which can be substituted or unsubstituted, such as hydroxy substituted.

Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “alkylaryl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heteroaryl” as used herein refers to mono- and polycyclic aromatic groups containing from 5 to 15 ring atoms wherein at least one ring atom is a heteroatom selected from nitrogen (N), oxygen (O), and sulfur(S). Heteroaryl groups can be monocyclic (5- or 6-membered ring) or bicyclic (8-, 9-, or 10-membered ring). Examples of monocyclic heteroaryl groups include but are not limited to furanyl, thiophenyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, and triazinyl groups. Examples of bicyclic heteroaryl groups include but are not limited to benzofuranyl, benzothiophenyl, benzoxazolyl, benzothiazolyl, benzimidazolyl, indolyl, isoindolyl, indazolyl, quinolinyl, isoquinolinyl, quinoxalinyl, quinazolinyl, imidazo[1,2-a]pyridinyl, triazyolyl, tetrazolyl, benzoxazolinyl, thiazolyl, benzthiazolinyl, and benzimidazolinyl groups.

Examples of benzoxazolinyl groups include groups having the general formula:

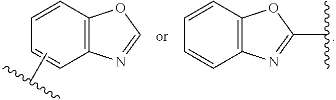

Examples of benzthiazolinyl groups include groups having the general formula:

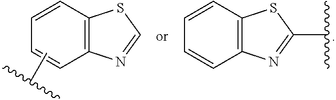

Examples of imidazo[1,2-a]pyridinyl groups include groups having the general formula:

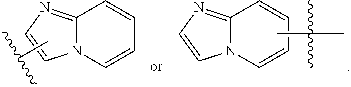

Representative substituted heteroaryl groups can be optionally substituted one or more times with substituents selected from amino, hydroxy, cyano, carboxy, nitro, thio, alkoxy, and halogen groups. In some embodiments, the heteroaryl rings may be optionally substituted as defined above. In other embodiments, optional substitution includes N—H substitution of the heteroaryl ring. For example, the heteroaryl ring may be optionally substituted with alkyl, alkenyl, alkynyl, heterocyclyl, aryl, heteroaryl, or other substituents as described herein.

The term “heterocyclyl” or “heterocyclo” refers to substituted or unsubstituted aromatic and non-aromatic ring compounds containing 3 or more ring members, of which one or more (e.g., 1, 2 or 3) is a heteroatom such as, but not limited to, N, O, and S. Thus, a heterocyclyl can be a heterocycloalkyl or a heteroaryl or, if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C₄-heterocyclyl can be a 5-membered ring with one heteroatom, a 6-membered ring with two heteroatoms, and so forth. The number of carbon atoms plus the number of heteroatoms equals the total number of ring atoms. Examples of heterocyclyl groups include, but are not limited to, azetidinyl, piperidynyl, piperazinyl, morpholinyl, pyrrolidinyl, or pyrrolidinone (e.g., the radical of pyrrolidin-2-one) groups.

A heterocyclyl ring can also include one or more double bonds, such as in the group 3,6-dihydro-2H-pyran and 3,4-dihydro-2H-pyran, having the formula:

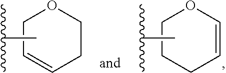

respectively, each of which may be substituted. Other representative heterocyclyl groups include, but are not limited to, tetrahydro-2H-thiopyran-1,1-dioxide, having the formula:

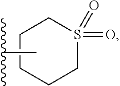

which may be substituted.

The term “heterocyclyl group” also includes fused ring species including those that include bicyclic groups. An example of a bicyclic heterocyclyl group is the 4a,5,6,7-tetrahydro-4H-pyrrolo[1,2-d][1,3,4] oxadiazinyl group, having the general formula:

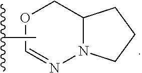

The term “heterocyclyl group” further includes fused ring species including fused aromatic and non-aromatic groups. Representative groups include, but are not limited to, tetrahydrobenzofuranyl, benzodioxolyl, chromanyl (eg. a radical of 3,4-dihydro-2H-chromene), chromanonyl, indolinonyl, isoindolinonyl, and 4a,5,6,7-tetrahydro-4H-pyrrolo[1,2-d][1,3,4]oxadiazinyl:

Examples of tetrahydrobenzofuranyl groups include groups having the general formula:

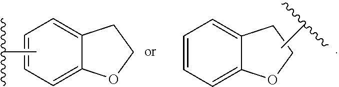

Examples of benzodioxlyl groups include groups having the general formula:

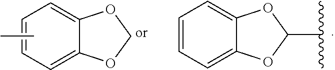

Examples of chromanyl groups include groups having the general formula:

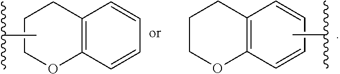

Examples of chromanonyl groups include groups having the general formula:

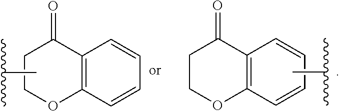

Examples of indolinonyl groups include groups having the general formula:

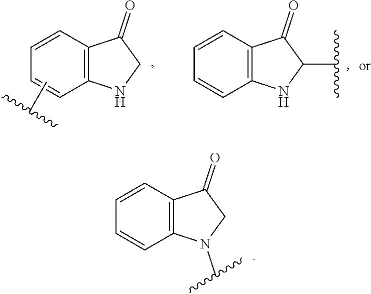

Examples of isoindolinonyl groups include groups having the general formula:

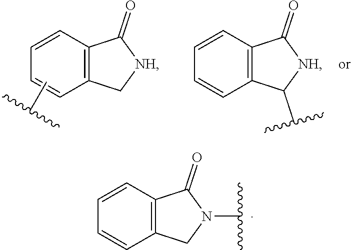

The aromatic and non-aromatic portion of the heterocyclyl group may both contain a heteroatom, for example, 3,4-dihydro-2H-1λ-1,8-naphthyridine, having the general formula:

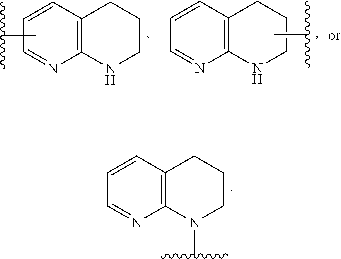

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂) morpholinyl such as —O—CH₂CH₂-morpholine.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” refer to a substituent of the form —NH₂, —NHR, —NR′R″, or —N′RR″R′″, wherein each R, R′, R″ and R′″ are defined herein, and protonated forms of each, except for-N′RR″R′″, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The term “amido” refers to a group of the formula —C(O)NR₂, wherein R is defined herein.

The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

An “effective amount” refers to any amount that is sufficient to achieve a desired biological effect. Combined with the teachings provided herein, by choosing among the various active conjugates or compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

A “therapeutically effective amount” (or “effective amount”) of a compound with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

For any compound a therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. A pharmaceutical preparation for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, and sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N. Y., pp. 367-383 (1981); Newmark et al., J Appl Biochem 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

The compound can be included in the formulation as fine multi-particulates in the form of granules or pellets of particle size about 1 mm. The formulation of the material for capsule administration could also be as a powder, lightly compressed plugs or even as tablets. Therapeutic agent could be prepared by compression.

Colorants and flavoring agents may all be included. For example, the compound can be formulated (such as by liposome or microsphere encapsulation) and then further contained within an edible product, such as a refrigerated beverage containing colorants and flavoring agents.

One may dilute or increase the volume of the compound with an inert material. These diluents can include carbohydrates, especially mannitol, α-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Disintegrants can be included in the formulation of therapeutic agent into a solid dosage form Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders, and these can include powdered gums such as agar, Karaya or tragacanth. Alginic acid and its sodium salt are also useful as disintegrants.

Binders can be used to hold the compound together to form a hard tablet and can include materials from natural products such as acacia, tragacanth, starch and gelatin. Others include methyl cellulose (MC), ethyl cellulose (EC) and carboxymethyl cellulose (CMC). PVP and hydroxypropylmethyl cellulose (HPMC) can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents, which can be used, include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added Microspheres formulated for oral administration can also be used. Such microspheres have been well-defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator, can be formulated containing a powder mix of the compound and a suitable powder base, such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (al-antitrypsin); Smith et al., 1989, J Clin Invest 84:1145-1146 (α-1-proteinase); Oswein et al., 1990, “Aerosolization of Proteins,” Proceedings of Symposium on Respiratory Drug Delivery II, Keystone, Colorado, March, (recombinant hepatocyte growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same), issued Sep. 19, 1995, to Wong et al.

Contemplated for use are a wide range of mechanical devices designed for pulmonary delivery of therapeutic products including, but not limited to, nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosolized, pelleted for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R. Science 249:1527-1533 (1990).

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances, which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction, which would substantially impair the desired pharmaceutical efficiency.

Therapeutic agent(s), including specifically, but not limited to, a compound, can be provided in particles. “Particles” means nanoparticles or microparticles (or in some instances larger particles) that can consist in whole or in part of the compound or the other therapeutic agent(s). The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:5823-2787 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Therapeutic agent(s) can be contained in controlled-release systems. The term “controlled release” refers to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) refers to a drug formulation that provides for gradual release of a drug over an extended period of time, and that can result in substantially constant blood levels of a drug over an extended time period. The term “delayed release” refers to a drug formulation in which there is a time delay between administration of the formulation and the release of the drug therefrom. “Delayed release” may or may not involve gradual release of drug over an extended period of time, and thus may or may not be “sustained release.”

Use of a long-term, sustained-release implant can be particularly suitable for treatment of chronic conditions. “Long-term” release means that the implant is constructed and arranged to deliver therapeutic levels of the active ingredient for at least 7 days, and up to 30-60 days. Long-term sustained-release implants are well-known to those of ordinary skill in the art and include some of the release systems described above.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, the disclosure of which is hereby incorporated by reference.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt: (6) gelatin; (7) talc; (8) excipients, such as cocoa butter and suppository waxes; (9) oils, such as peanut oil, cottonseed oil, safflower oil, sesame oil, olive oil, corn oil and soybean oil: (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “administering” includes all means of introducing the compounds and compositions described herein to the patient, including, but are not limited to, oral (po). intravenous (iv), intramuscular (im), subcutaneous (sc), transdermal, inhalation, buccal, ocular, sublingual, vaginal, rectal, and the like. The compounds and compositions may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well-known to those skilled in the art. Parenteral administration of a compound is illustratively performed in the form of saline solutions or with the compound incorporated into liposomes. In cases where the compound, itself, is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

In the methods the individual components of a co-administration, or combination, can be administered by any suitable means, contemporaneously, simultaneously, sequentially in either order, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well-known to the researcher, veterinarian, medical doctor or other clinician of ordinary skill.

Depending upon the route of administration, a wide range of permissible dosages are contemplated, including doses falling in the range from about 1 μg/kg to about 1 g/kg. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases the described therapeutically effective amounts correspond to the instance of administration, or alternatively to the total daily, weekly, month, or quarterly dose, as determined by the dosing protocol.

An “effective amount” of any one or a mixture of the compounds can be determined by the attending diagnostician or physician by the use of known techniques and/or by observing results obtained under analogous circumstances. In determining the effective amount or dose, a number of factors are considered by the attending diagnostician or physician, including, but not limited to, the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human. The term “Lewy body” refers to an abnormal intracellular inclusion primarily composed of aggregated, misfolded alpha-synuclein protein, often accompanied by ubiquitin and other cellular components. Lewy bodies are characteristic of certain neurodegenerative disorders, such as Parkinson's disease and dementia with Lewy bodies, and are typically localized in brain regions associated with motor control, cognition, and other neurological functions.

The term “alpha-synuclein” (α-syn) refers to a small, soluble protein primarily expressed in the nervous system, where it is abundant in presynaptic terminals. Alpha-synuclein (α-syn) plays a role in synaptic vesicle trafficking and neurotransmitter release under normal physiological conditions. In pathological states, α-syn can misfold and aggregate, forming insoluble fibrils that are the main component of Lewy bodies, a hallmark of neurodegenerative diseases such as Parkinson's disease and dementia with Lewy bodies.

The term “Parkinson's disease” refers to a neurodegenerative disorder characterized by the progressive loss of dopaminergic neurons in the substantia nigra pars compacta and the pathological accumulation of alpha-synuclein (α-syn) in the form of Lewy bodies. Parkinson's disease is associated with motor symptoms such as bradykinesia, rigidity, resting tremor, and postural instability, as well as non-motor symptoms, including but not limited to autonomic dysfunction, cognitive impairment, and mood disorders.

The term “dementia with Lewy bodies (DLB)” refers to a progressive neurodegenerative disorder characterized by cognitive decline that interferes with daily functioning and is accompanied by fluctuating attention, visual hallucinations, and motor symptoms similar to Parkinson's disease. Pathologically, DLB is defined by the presence of Lewy bodies-intracellular aggregates primarily composed of misfolded alpha-synuclein (α-syn)-in cortical and subcortical regions of the brain. It is considered one of the most common causes of dementia, distinct from Alzheimer's disease by its clinical and pathological features.

The term “multiple system atrophy (MSA)” refers to a rare, progressive neurodegenerative disorder characterized by autonomic dysfunction, parkinsonism, and cerebellar ataxia. Pathologically, MSA is defined by the accumulation of misfolded alpha-synuclein (o-syn) in oligodendrocytes, forming glial cytoplasmic inclusions (GCIs). The disease affects multiple systems, including motor and autonomic pathways, and is subdivided into two main types based on the predominant symptoms: MSA-P (parkinsonian subtype) and MSA-C (cerebellar subtype). The term “AA amyloidosis” refers to a systemic disorder caused by the extracellular deposition of amyloid fibrils composed of misfolded serum amyloid A (SAA) protein. It is often secondary to chronic inflammatory conditions or infections and primarily affects organs such as the kidneys, liver, and spleen, leading to progressive organ dysfunction.

The term “Alzheimer's disease” refers to a progressive neurodegenerative disorder characterized by cognitive decline, memory impairment, and behavioral changes. Pathologically, it is defined by the accumulation of extracellular amyloid-beta plaques and intracellular tau neurofibrillary tangles in the brain, along with neuronal loss and synaptic dysfunction.

The term “monoclonal immunoglobulin light-chain amyloidosis” (AL amyloidosis) refers to a disorder caused by the deposition of misfolded monoclonal light chains derived from clonal plasma cells. These misfolded proteins form insoluble amyloid fibrils that accumulate in various tissues, leading to progressive organ damage, particularly in the heart, kidneys, liver, and peripheral nerves.

The term “Huntington's disease” refers to a genetic neurodegenerative disorder caused by an expanded CAG trinucleotide repeat in the HTT gene, leading to the production of mutant huntingtin protein. The disease is characterized by progressive motor dysfunction, psychiatric symptoms, and cognitive decline, with pathological hallmarks including neuronal loss and intracellular inclusions of aggregated huntingtin protein.

The term “Parkinson's disease” refers to a neurodegenerative disorder characterized by the progressive loss of dopaminergic neurons in the substantia nigra pars compacta and the pathological accumulation of alpha-synuclein (α-syn) in the form of Lewy bodies. Parkinson's disease is associated with motor symptoms such as bradykinesia, rigidity, resting tremor, and postural instability, as well as non-motor symptoms, including but not limited to autonomic dysfunction, cognitive impairment, and mood disorders.

The term “Creutzfeldt-Jakob disease” (CJD) refers to a rare, fatal neurodegenerative disorder caused by the accumulation of misfolded prion protein (PrPASc), which induces the aggregation of normal prion protein (PrPAC) in the brain. CJD is characterized by rapidly progressive dementia, motor dysfunction, and myoclonus, with spongiform changes observed in the brain tissue upon histological examination.

The term “prion disorders” refers to a group of fatal neurodegenerative diseases caused by the misfolding and aggregation of prion protein (PrP), leading to the propagation of the pathological isoform (PrPASc). These disorders include Creutzfeldt-Jakob disease, fatal familial insomnia, Gerstmann-Sträussler-Scheinker syndrome, and kuru, all of which are characterized by spongiform changes, neuronal loss, and gliosis.

The term “amyotrophic lateral sclerosis” (ALS) refers to a progressive neurodegenerative disorder characterized by the loss of motor neurons in the brain and spinal cord, leading to muscle weakness, atrophy, and eventual paralysis. Pathological features include the aggregation of proteins such as TDP-43 and, in some cases, SODI or FUS, which contribute to motor neuron degeneration.

The term “type 2 diabetes” refers to a metabolic disorder characterized by chronic hyperglycemia due to insulin resistance and relative insulin deficiency. Pathophysiological features include impaired glucose uptake, pancreatic beta-cell dysfunction, and amyloid deposits composed of islet amyloid polypeptide (IAPP) in the pancreatic islets.

The term “transthyretin amyloidosis” (ATTR) refers to a systemic disorder caused by the misfolding and aggregation of transthyretin (TTR) protein into amyloid fibrils. ATTR can manifest in hereditary or wild-type forms, with clinical symptoms including polyneuropathy, cardiomyopathy, and progressive organ dysfunction due to amyloid deposition.

The term “anti-aggregation compound” refers to a substance or molecule that inhibits or prevents the aggregation of proteins, peptides, or other biological macromolecules. Such compounds are designed to interfere with the processes that lead to the misfolding, clustering, or accumulation of these molecules, which are often associated with pathological conditions, including neurodegenerative diseases like Parkinson's disease, Alzheimer's disease, and multiple system atrophy. Anti-aggregation compounds may act by stabilizing native protein conformations, disrupting aggregation-prone intermediates, or disassembling existing aggregates.

The disclosure also relates to the following numbered Embodiments presented in no particular order of importance:

1. A compound of the formula (Ia) or (Ib):

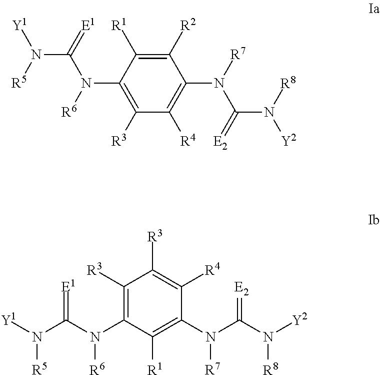

or a pharmaceutically acceptable salt thereof,
wherein:
E¹ and E² are each independently O, S, or NH;
R¹-R⁴ are each independently H, halogen, C₁-C₂₀ alkyl, or C₁-C₂₀ alkoxy;
R⁵-R⁸ are each independently H or C₁-C₂₀ alkyl;
Y¹ and Y² are each independently C₃-C₈ cycloalkyl; or

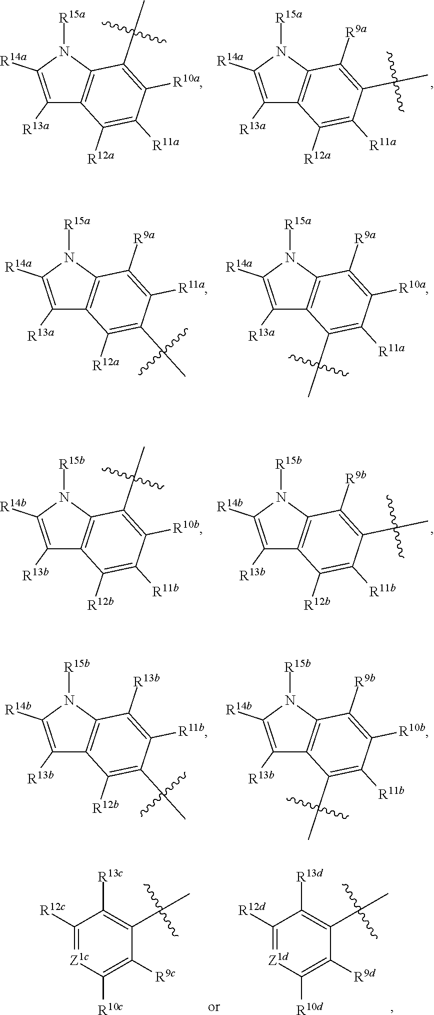

wherein
Z^{1c} is N or C—R^{11c};
Z^{1d} is N or C—R^{11d}
R^{9a}-R^{15a}, R^{9b}-R^{15b}, R^{9c}-R^{15c}, and R^{9d}-R^{15d} are each independently H, halogen, —OH, —NH₂, —NO₂, —CF₃, —CN, —COOH, —N₃, —SO—H, or —PO₃H₂, C₁-C₂₀ alkyl, C₁-C₂₀ alkoxy, C₂-C₂₀ alkenyl, C₂-C₂₀ alkynyl, C₃-C₁₂ cycloalkyl, C₃-C₁₂ cycloalkenyl, or 3- to 15-membered heterocyclyl group.
   2. A compound of the formula (Ic):

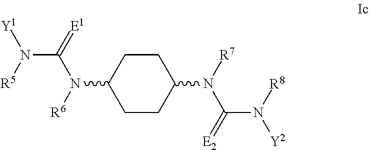

or a pharmaceutically acceptable salt thereof,
wherein:
E¹ and E² are each independently O, S, or NH;
R⁵-R⁸ are each independently H or C₁-C₂₀ alkyl;
Y¹ and Y² are each independently:

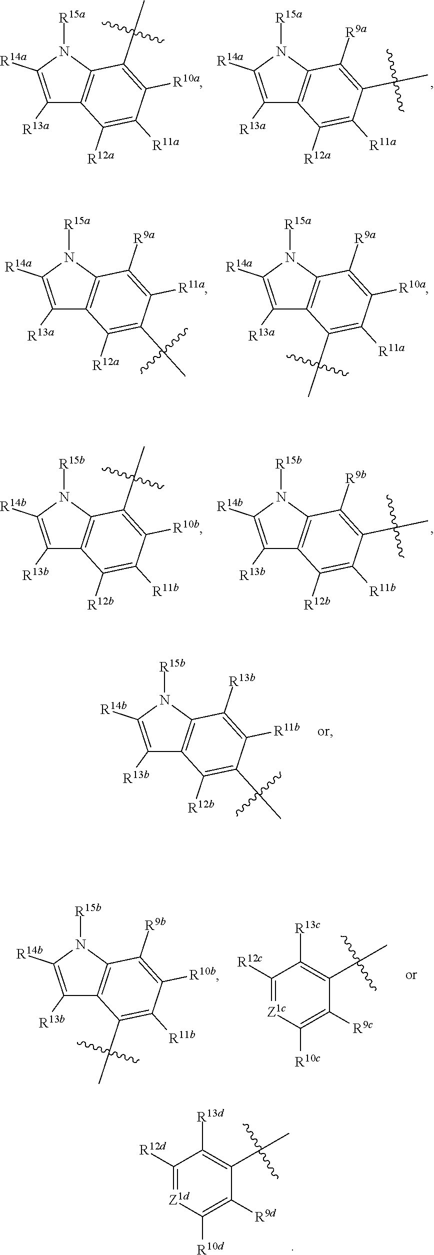

wherein
Z^{1c} is N or C—R^{11c};
Z^{1d} is N or C—R^{11d}
R^{9c}-R^{15a}, R^{9b}-R^{15b}, R^{9c}-R^{15c}, and R^{9d}-R^{15d} are each independently H, halogen, —OH, —NH₂, —NO₂, —CF₃, —CN, —COOH, —N₃, —SO₃H, or —PO₃H₂, C₁-C₂₀alkyl, C₁-C₂₀ alkoxy, C₂-C₂₀ alkenyl, C₂-C₂₀ alkynyl, C₃-C₁₂ cycloalkyl, C₃-C₁₂ cycloalkenyl, or 3- to 15-membered heterocyclyl group.
   3. The compound of Embodiment 2 having the structure

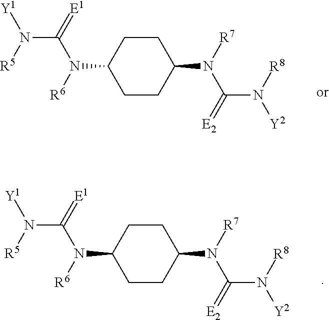

4. The compound of any one of Embodiments 1-3, wherein E¹ and E² are both O.
5. The compound of any one of Embodiments 1-3, wherein E¹ and E² are both S.
6. The compound of any of Embodiments 1-5, wherein R¹-R⁴ are H.
7. The compound of any of Embodiments 1-5, wherein R¹ is alkyl and R²-R⁴ are H.
8. The compound of Embodiment 7, wherein R¹ is methyl.
9. The compound of any of Embodiments 1-7, wherein R⁵ and R⁶ are each independently H.
10. The compound of any of Embodiments 1-8, wherein R⁷ and R⁸ are each independently H.
11. The compound of any of Embodiments 1-9, wherein R⁵-R⁸ are H.
12. The compound of any of Embodiments 1-3, wherein Y¹ and Y² are each independently

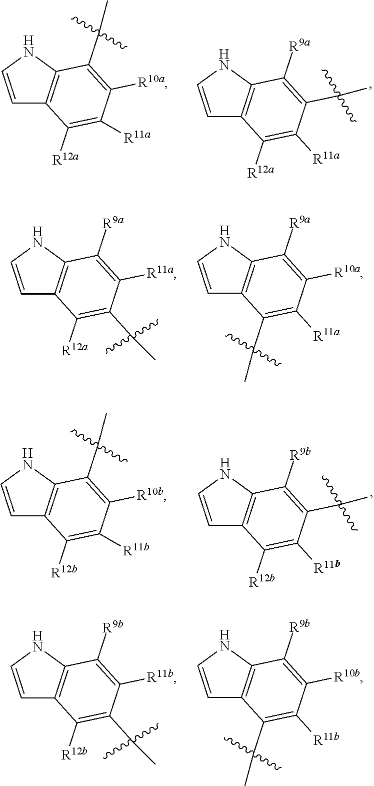

wherein R^{9c}-R^{15c} and R^{9b}-R^{15b} are each independently H, halogen or C₁-C₆ alkyl;
or a pharmaceutically acceptable salt thereof.
   13. The compound of Embodiment 12, wherein both Y¹ and Y² are

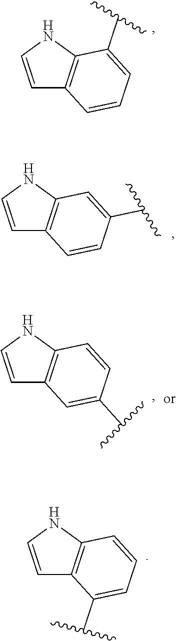 [SMILES: *c1cccc2cc[nH]c12 ; *c1ccc2cc[nH]c2c1 ; *c1ccc2[nH]ccc2c1 ; *c1cccc2[nH]ccc12]

14. The compound of any of Embodiments 1-3, wherein Y¹ and Y² are each independently

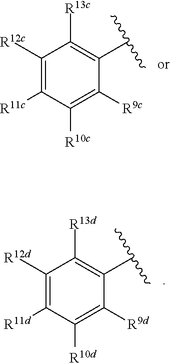

15. The compound of any of Embodiments 1-3, wherein R^{11c} and R^{11d} are each independently a 6-membered heterocycle.
16. The compound of any of Embodiments 1-3, wherein Y¹ and Y² are each independently

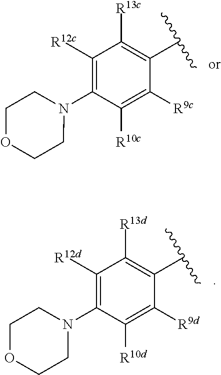

17. The compound of any of Embodiments 1-3, wherein Y¹ and Y² are each independently

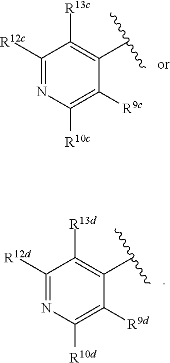

18. A compound of the formula:

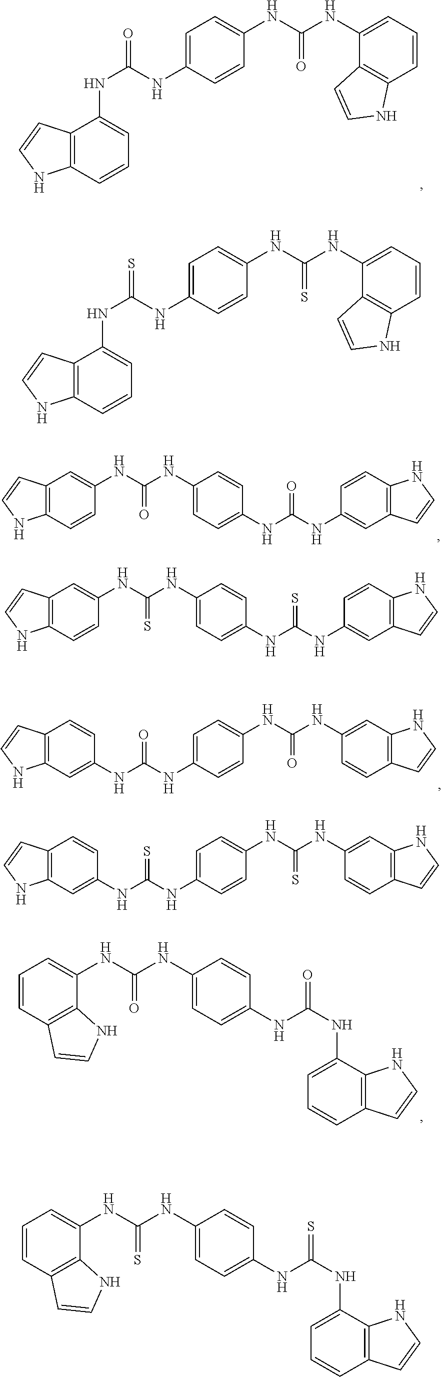

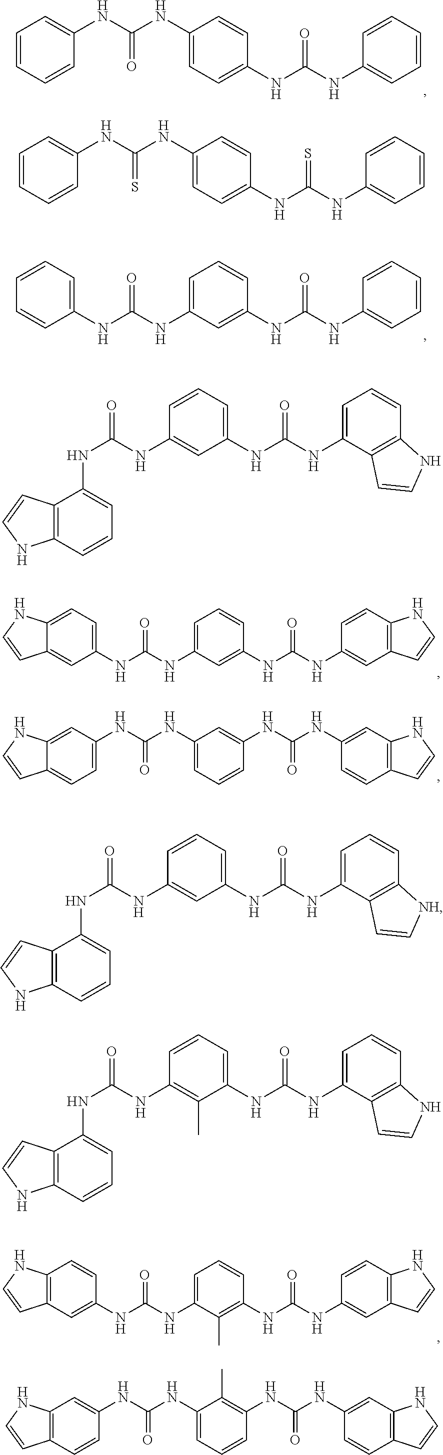

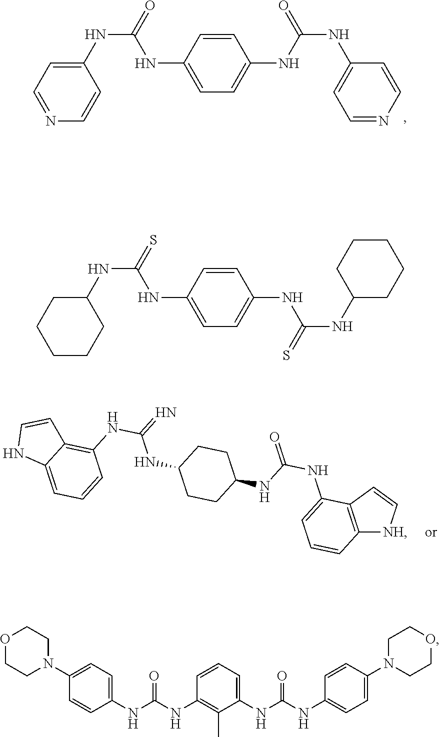 [SMILES: O=C(Nc1ccncc1)Nc1ccc(NC(=O)Nc2ccncc2)cc1 ; S=C(Nc1ccc(NC(=S)NC2CCCCC2)cc1)NC1CCCCC1 ; N=C(Nc1cccc2[nH]ccc12)N[C@H]1CC[C@H](NC(=O)Nc2cccc3[nH]ccc23)CC1 ; Cc1c(NC(=O)Nc2ccc(N3CCOCC3)cc2)cccc1NC(=O)Nc1ccc(N2CCOCC2)cc1]

or a pharmaceutically acceptable salt thereof.
   19. A pharmaceutical composition comprising the compound of any one of Embodiments 1-18 and a pharmaceutically acceptable carrier.
   20 A method of inhibiting α-synuclein (α-syn) protein aggregation in a subject having, or at risk for, α-syn protein aggregation, comprising administering a compound of any one of Embodiments 1-18 or a pharmaceutical composition of Embodiment 19 to the subject in need thereof.
   21. The method of Embodiment 20, wherein the subject has, or is at risk for, Alzheimer's disease.
   22. The method of Embodiment 20, wherein the subject has, or is at risk for, dementia with Lewy bodies (DLB).
   23. The method of Embodiment 20, wherein the subject has, or is at risk for, multiple system atrophy (MSA).
   24 The method of Embodiment 20, wherein the subject has neuroblastoma and formation of α-syn inclusions is inhibited.

### EXAMPLES

The following examples serve to illustrate the present disclosure. The examples are not intended to limit the scope of the claimed invention in any way.

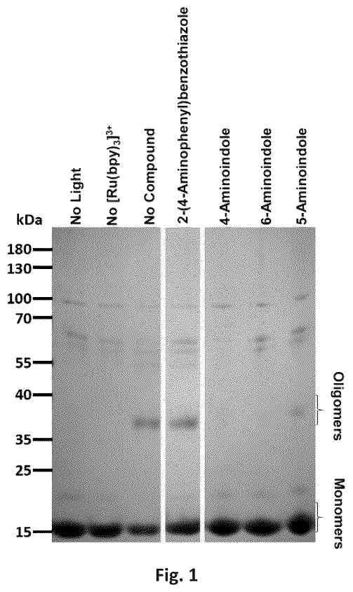

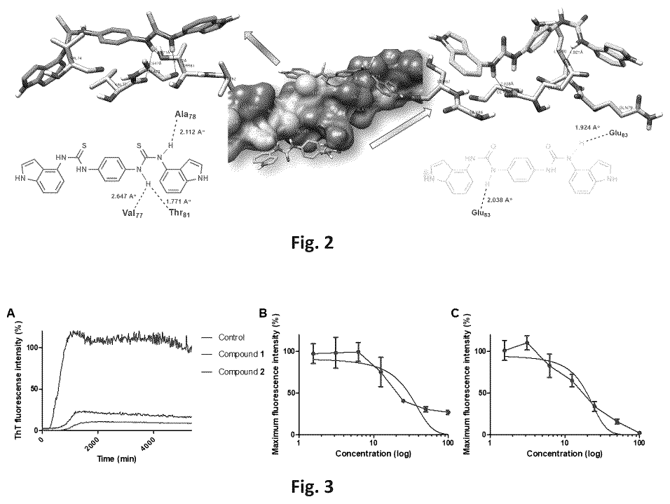

Chemical and peptide source. Dimethylsulfoxide (DMSO) and thioflavin-T (ThT) were purchased from Alfa Aesar (Ward Hill, MA). Recombinant α-synuclein (α-syn) was procured from rPeptide (WatKinsville, GA). The recombinant p-tau isoform 1N4R was prepared as published previously (Fortin et al. 2021, Sui et al. 2015, Liu et al. 2020). Concerning the preparation of tau 0N4R, a bacterial expression plasmid consisting of the vector pET30a carrying a cDNA encoding the human Tau 0N4R isoform was a kind gift of Dr. Benjamin Wolozin (Boston University). E. coli stock (Rosetta BL21 E. coli (CamR) containing pET30a [0N4R tau wt] (KanR)) was grown in LB medium supplemented with kanamycin (50 μg/mL) and chloramphenicol (50 μg/mL). Protein over-expression was induced by the addition of 1 mM IPTG for ˜18 hours at 37° C., and cells were pelleted by centrifugation at 6,000 g for 15 minutes at 4° C. The cells were resuspended in lysis buffer (10 mM Hepes, pH 7.4, 50 mM NaCl, 1 mM MgCl₂, 1 mM PMSF, 1× PIC, and 0.5 mM DTT) and lysed by sonication at 30 seconds “On” and 1 minute “Off” at ˜30-45% power for ˜3-5 minutes. Lysate was centrifuged at 10,000 rpm for 10 minutes at 4° C., and supernatant was transferred with 7.8 ml (total) of 3M NaCl. The lysate was incubated for 10 minutes in 80° C. water bath, then cooled for 10 minutes in an ice bath. The lysate was centrifuged at 10,000 rpm at 4° C. for 10 minutes, and the supernatant was transferred to a new tube. The supernatant was dialyzed overnight against cation exchange buffer (50 mM MES, 1M NaCl, 1 mM DTT, pH 6.0). The dialysate was loaded onto a HiPrep SP HP column, and proteins were eluted with a linear gradient ranging from 50 mM to 1 M NaCl.
General Characterizations. ¹H and ¹³C {1H} NMR spectra were recorded using a 500 MHz Bruker instrument working at a frequency of 500 MHz for ¹H and at 126 MHz for ¹³C. Chemical shifts are reported in ppm using residual solvent resonances as internal reference (δ 2.50 and δ 39.51 for ¹H and ¹³C in DMSO-d₆, respectively). ¹H NMR data are reported as follows. b=broad, s=singlet, d=doublet, t=triplet, q=quartet, and m=multiplet. Coupling constants are given in hertz. The purity of all compounds and synthetic intermediates was judged to be 95% or better based on ¹H NMR. IR measurements were performed in a Nicolet FTIR as thin films in the Purdue Drug Discovery facility. High-resolution mass spectrometry analyses were conducted at the MSU Mass Spectrometry facility.
Thioflavin fluorescence assays. Thioflavin fluorescence assays were used to monitor fibril formation of commercial recombinant α-syn at a final concentration of 2 μM and treated with control DMSO and different compounds. The recombinant α-syn obtained from rPeptide has been validated with proper quality control to confirm the monomeric state of the protein. Kinetics of fibril formation have been performed using ThT as published previously.^{6, 12, 13, 24} The fluorescence emission experiments were performed with the excitation and emission wavelengths set at 440 and 485 nm, respectively, with a Synergy HT multi-mode microplate reader (BioTek, Winooski, VT). Samples were measured in three replicates. Experiments were repeated three times using different α-syn stock solutions. For each time point, arbitrary units of fluorescence were calculated from the mean values normalized against the maximum value. The percentage of fluorescence intensity at the plateau phase in Table 1 was expressed as mean #SEM. Concerning the dose response curve depicted in FIG. 2, the data were plotted using GraphPad Prism.
Photo-induced Cross-linking of Unmodified Proteins (PICUP) Assay. To induce oligomerization by cross-linking, α-syn (from Rpeptide, LLC) was diluted in 10 mM phosphate buffer (pH 7.4) to reach a final concentration of 50 μM. Different compounds were added to the protein solution at a final concentration of 50 μM, resulting in a molar ratio of 1:1. PICUP assays were performed with the following controls: samples without light exposition, without Ru(bpy) or ammonium persulfate, and without compound (i.e., 0.125% DMSO). The addition of 2 μL of Ru(bpy) (300 μM final concentration) and 2 μL ammonium persulfate (6 mM final concentration) to the samples is required prior to light exposure.^{6, 12, 13} Light exposure was of a 1 second duration for α-syn with a 53 W (120 V) incandescent lamp installed in a homemade dark-box. Each tube contained a final volume of 20 μL. To end the radical reaction, 8.3 μL of Laemmli loading buffer containing 15% β-mercaptoethanol was added to the protein solution and subjected to incubation at 95° C. for 10 minutes. The cross-linked α-syn samples were separated using a 16% SDS-PAGE gel and then stained with Coomassie blue staining.
Transmission electron microscopy (TEM). TEM was utilized to detect fibril formation at the end of ThT kinetics of fibril formation as mentioned in a previous publication.^{6, 12, 13} A volume of 10 μL was applied on a 400-mesh Formvar-carbon-coated copper grid (Electron Microscopy Sciences, Hatfield, PA). The grids were incubated in contact with α-syn samples for one minute. Grids were washed three times with distilled water. They were carefully air-dried and incubated for one minute in a fresh solution of 1% uranyl acetate. After removal of excess of stain, acquisition of pictures was performed with the following settings using a transmission electron microscope (JEOL 1400 Flash, Japan): accelerating voltage of 100 kV and magnification of 40 k.
α—Syn (or αS) inclusion-forming neuroblastoma cell experiment. Dox-inducible neuroblastoma cells M17D-TR/αS-3K::YFP have been used previously.²¹ 96-well plates were used with cellular density of 30,000 cells per well. Compounds were added after 24 hours and αS-3K::YFP transgene expression was induced 48 hours later. Induction was done by adding 1 μg per mL (final concentration) dox to culture media. Cells were incubated in the Incucyte Zoom 2000 platform (Essen Biosciences) and images (green, bright field) were taken continuously. Endpoint analysis of inclusion formation or growth was performed 48 hours after induction (96 hours after plating). The Incucyte processing definition ‘Inclusions’ was created as follows: Parameters, Fixed Threshold, Threshold (GCU) 50; Edge Split On, Edge Sensitivity 100; Cleanup, Hole Fill (μm²): 10, Adjust Size (pixels): 0; Filters, Area (μm²): max 50, Mean Intensity: min 60, Integrated Intensity: min 2000. Cell confluence was measured by the processing definition ‘Cells’: Parameters, Segmentation Adjustment 0.7; Cleanup, all parameters set to 0; Filters, Area (μm²): min 345.00. As described previously for the evaluation of protein expression by SDS-PAGE and Western Blotting in the LiCor system, αS-specific monoclonal antibody 4B12 (Thermofisher, Waltham, MA; 1:1000) and a polyclonal antibody to GAPDH (Sigma-Aldrich, St. Louis, MO, G9545; 1:5000) were used.²²
Docking Studies. The geometries of the ligands were optimized using Becke's three-parameter hybrid exchange functional along with the Lee-Yang-Parr correction functional (B3LYP) method in conjunction with the 6-311G+(d, p) basis set using the Gaussian 16 software package. The molecular docking was performed with AutoDock 4.0 (Version 1.5.7). The grid size was set to 100×100×100 points with a grid spacing of 0.375 A° to cover the reported active site (Val3, Lys10, Ala17, Lys21, Ala69, Val70, Gly73, Ala76, Val77, Lys80, Glu83, Gly84, Ser87) of human alpha-synuclein protein (PDB ID: 1XQ8) containing 140 amino acids (Selvaraj et al. 2021). The intermolecular polar interactions and bond angles were analyzed and visualized by UCSF Chimera (version 1.14).
Synthesis. Aminoindoles were selected as main substituents of the 1,4-diurea and 1,4-dithiourea analogs based on their anti-oligomer activity demonstrated by PICUP assay (FIG. 1). The synthesis of diurea (1, 3, 5, 7, and 9) and its bioisosteric dithiourea analogs (2, 4, 6, 8, and 10) is shown in Scheme 1. The synthesis of analogs was achieved via nucleophilic addition of the corresponding amines (a-e in Table 1), mainly four aminoindoles and one aniline, with the respective diisocyanate (P) or diisothiocyanate (Q) as shown in Table 1 and Scheme 1. All the analogs have TPSA in the range of 79-114 Å² with <4 hydrogen bond donors (HBD), which obeyed Lipinski's rule.

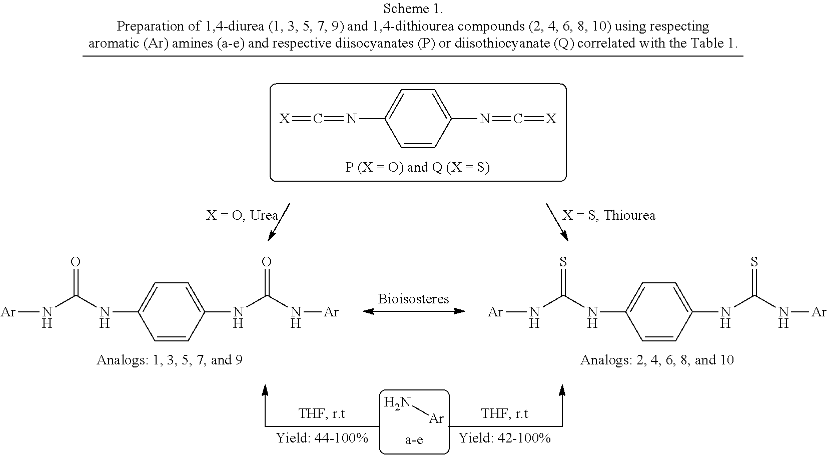

### General Synthetic Procedure (1-10)

Respective aromatic anilines a-e (2.0 equiv.) were dissolved in THF at room temperature. The reaction mixture was charged with a respective diisocyanate (P, 1.0 equiv.) or diisothiocyanate (Q, 1.0 equiv.) at room temperature. The reaction mixture was stirred at room temperature until all the starting materials were consumed as monitored by TLC. The resultant precipitate was filtered and washed thoroughly with hexane:dichloromethane (1:1) until it removed all the amine traces to produce pure respective diurea (1, 3, 5, 7, 9) or dithiourea (2, 4, 6, 8, 10) analogs.

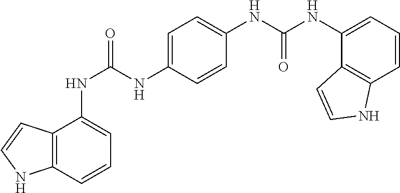 [SMILES: O=C(Nc1ccc(NC(=O)Nc2cccc3[nH]ccc23)cc1)Nc1cccc2[nH]ccc12]

3-(1H-indol-4-yl)-1-(4-{[(1H-indol-4-yl)carbamoyl]amino}phenyl)urea (1): White color solid (452 mg, yield: quantitative), R_{f}=0.22 (dichloromethane/MeOH=95:5). ¹H NMR (500 MHZ, DMSO) δ 11.11 (s, 1H), 8.79 (s, 1H), 8.43 (s, 1H), 7.67 (dd, J=7.5, 1.1 Hz, 1H), 7.44 (s, 2H), 7.31 (t, J=2.8 Hz, 1H), 7.06-6.99 (m, 2H), 6.57 (s, 1H). ¹³C {¹H} NMR (126 MHz, DMSO) δ 152.6, 136.5, 134.1, 132.7, 124.0, 121.6, 118.8, 107.3, 105.7, 97.7. m.p.<355° C. HRMS (ESI/Q-TOF) m/z: [M+H]⁺ Calcd. for C₂₄H₂₀N₆O₂ 425.1728; Found 425.1729. IR (cm⁻¹) v=3607, 3315, 1668, 1624, 1559, 1506, 1407, 1301, 1229, 1201, 746, 721.

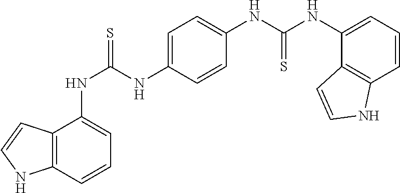 [SMILES: S=C(Nc1ccc(NC(=S)Nc2cccc3[nH]ccc23)cc1)Nc1cccc2[nH]ccc12]

3-(1H-indol-4-yl)-1-(4-{[(1H-indol-4-yl)carbamothioyl]amino}phenyl)thiourea (2): Ash color solid (286 mg, yield: 63%), R_{f}=0.44 (dichloromethane/MeOH=95:5). ¹H NMR (500 MHZ, DMSO) δ 11.20 (s, 2H), 9.77 (s, 2H), 9.52 (s, 2H), 7.35 (s, 4H), 7.34 (t, J=2.8 Hz, 2H), 7.27 (dd, J=16.5, 7.8 Hz, 4H), 7.07 (t, J=7.8 Hz, 2H), 6.46 (s, 2H). ¹³C {¹H} NMR (126 MHz, DMSO) δ 179.1, 137.0, 136.1, 130.6, 125.0, 123.8, 122.9, 121.0, 114.6, 109.0, 99.1. m.p. 197.2-202.4° C. HRMS (ESI/Q-TOF) m/z: [M+H]⁺ Calcd. for C₂₄H₂₀N₆S₂ 457.1271; Found 457.1278. IR (cm⁻¹) v=3313, 3148, 2985, 1617, 1537, 1501, 1413, 1343, 1257, 1217.

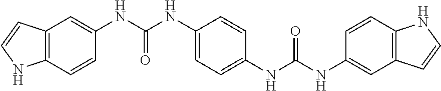 [SMILES: O=C(Nc1ccc(NC(=O)Nc2ccc3[nH]ccc3c2)cc1)Nc1ccc2[nH]ccc2c1]

3-(1H-indol-5-yl)-1-(4-{[(1H-indol-5-yl)carbamoyl]amino}phenyl)urea (3): Pale brown color solid (380 mg, yield: 45%), ¹H NMR (500 MHZ, DMSO) δ 10.91 (s, 1H), 8.36 (d, J=40.2 Hz, 2H), 7.66 (s, 1H), 7.31 (d, J=40.4 Hz, 4H), 7.07 (s, 1H), 6.34 (s, 1H). ¹³C {1H} NMR (126 MHZ, DMSO) δ 153.2, 134.2, 132.2, 132.6, 127.7, 125.7, 118.8, 114.7, 111.2, 109.8, 100.9. m.p. 317.0-323.4° C. IR (cm⁻¹) v=3315, 1614, 1559, 1505, 1474, 1406, 1309, 1220.

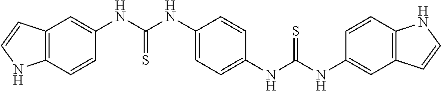 [SMILES: S=C(Nc1ccc(NC(=S)Nc2ccc3[nH]ccc3c2)cc1)Nc1ccc2[nH]ccc2c1]

3-(1H-indol-5-yl)-1-(4-{[(1H-indol-5-yl)carbamothioyl]amino}phenyl)thiourea (4): Off-white color solid (412 mg, yield: 92%), R_{f}=0.39 (dichloromethane/MeOH=95:5). ¹H NMR (500 MHz, DMSO) δ 11.09 (s, 2H), 9.62 (s, 2H), 9.42 (s, 2H), 7.55 (s, 2H), 7.42 (s, 4H), 7.71-7.35 (m, 4H), 7.07 (dd, J=8.6, 2.0 Hz, 2H), 6.43 (s, 2H). ¹³C {¹H} NMR (126 MHz, DMSO) δ 179.9, 136.1, 133.9, 130.6, 127.6, 126.1, 123.9, 119.5, 116.4, 111.3, 101.3. m.p. 187.3-191.6° C. IR (cm⁻¹) v=3315, 1668, 1624, 1559, 1506, 1407, 1301, 1229, 1201.

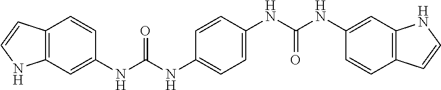 [SMILES: O=C(Nc1ccc(NC(=O)Nc2ccc3cc[nH]c3c2)cc1)Nc1ccc2cc[nH]c2c1]

3-(1H-indol-6-yl)-1-(4-{[(1H-indol-6-yl)carbamoyl]amino}phenyl)urea (5): Pale yellow color solid (364 mg, yield: 43%), ¹H NMR (500 MHz, DMSO) δ 10.89 (s, 1H), 8.49 (d. J=17.5 Hz, 2H), 7.90-7.68 (m, 1H), 7.39 (d, J=8.4 Hz, 1H), 7.36 (s, 2H), 7.25-7.14 (m, 1H), 6.84 (dd, J=8.5, 1.9 Hz, 1H), 6.32 (s, 1H). ¹³C {¹H} NMR (126 MHz, DMSO) δ 153.4, 136.8, 134.7, 134.4, 124, 8, 123.5, 120.4, 119.3, 112.4, 101.5, 101.3. m.p. 329.5-336.3° C. IR (cm⁻¹) v=3299, 1630, 1589, 1552, 1509, 1454, 1402, 1345, 1324, 1295, 1220.

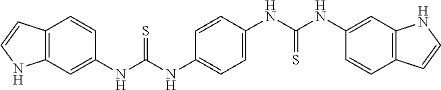 [SMILES: S=C(Nc1ccc(NC(=S)Nc2ccc3cc[nH]c3c2)cc1)Nc1ccc2cc[nH]c2c1]

3-(1H-indol-6-yl)-1-(4-{[(1H-indol-6-yl)carbamothioyl]amino}phenyl)thiourea (6): Off-white color solid (463 mg, yield: 100%), R_{f}=0.37 (dichloromethane/MeOH=95:5). ¹H NMR (500 MHz, DMSO) δ 11.07 (s, 2H), 9.74 (s, 2H), 9.55 (s, 2H), 7.61 (s, 2H), 7.49 (d, J=8.4 Hz, 2H), 7.44 (s, 4H), 7.32 (t, J=2.8 Hz, 2H), 6.97 (dd, J=8.5, 1.9 Hz, 2H), 6.40 (s, 2H). ¹³C {¹H} NMR (126 MHz, DMSO) δ 179.5, 136.0, 135.7, 132.8, 125.7, 125.2, 123.8, 119.8, 116.6, 107.2, 101.0. m.p. 221.1-225.4° C. IR (cm⁻¹) v=3312, 1618, 1537, 1500, 1409, 1343, 1255, 1217.

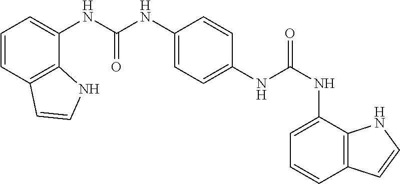 [SMILES: O=C(Nc1ccc(NC(=O)Nc2cccc3cc[nH]c23)cc1)Nc1cccc2cc[nH]c12]

3-(1H-indol-7-yl)-1-(4-{[(1H-indol-7-yl)carbamoyl]amino}phenyl)urea (7): Ash color solid (187 mg, yield: 44%), ¹H NMR (500 MHz, DMSO) δ 10.69 (s, 1H), 8.65 (s, 1H), 8.45 (s, 1H), 7.43 (s, 2H), 7.33 (t, J=2.8 Hz, 1H), 7.30 (d, J=7.8 Hz, 1H), 7.10 (d, J=7.6, 1H), 6.94 (t, J=7.7 Hz, 1H), 6.44 (dd, J=3.1, 1.9 Hz, 1H). ¹³C {¹H} NMR (126 MHZ, DMSO) δ 153.2, 134.2, 129.2, 128.4, 125.2, 123.9, 119.1, 119.0, 115.6, 113.4, 101.5. m.p.<360° C. IR (cm⁻¹) v=3397, 3275, 1634, 1572, 1537, 1504, 1435, 1343, 1236, 1206, 717, 661.

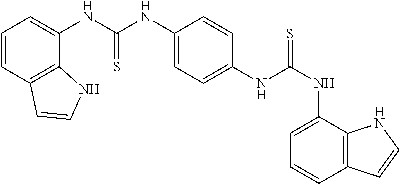 [SMILES: S=C(Nc1ccc(NC(=S)Nc2cccc3cc[nH]c23)cc1)Nc1cccc2cc[nH]c12]

3-(1H-indol-7-yl)-1-(4-{[(1H-indol-7-yl)carbamothioyl]amino}phenyl)thiourea (8): Off-white color solid (76 mg, yield: 42%), R_{f}=0.24 (hexane/ethyl acetate=7:3). ¹H NMR (500 MHZ, DMSO) δ 10.97 (s, 2H), 9.69 (s, 2H), 9.50 (s, 2H), 7.50 (s, 4H), 7.43 (d, J=7.7 Hz, 2H), 7.31 (t, J=2.8 Hz, 2H), 7.06 (d, J=8.0 Hz, 1H), 6.98 (t, J=7.6 Hz, 1H), 6.46 (dd, J=3.1, 1.9 Hz, 1H). ¹³C {¹H} NMR (126 MHz, DMSO) δ 180.2, 136.0, 131.6, 129.4, 125.5, 123.9, 123.6, 118.8, 118.2, 109.2, 101.5. m.p. 193.4-197.1° C. IR (cm⁻¹) v=3312, 3151, 1539, 1502, 1435, 1413, 1343, 1263, 1220, 744, 723.

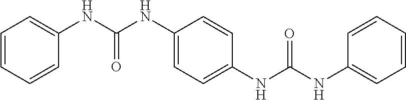 [SMILES: O=C(Nc1ccccc1)Nc1ccc(NC(=O)Nc2ccccc2)cc1]

1-phenyl-3-{4-[(phenylcarbamoyl)amino]phenyl}urea (9): White color solid (485 mg, yield: 93%), ¹H NMR (500 MHz, DMSO) δ 8.62 (s, 2H), 8.55 (s, 2H), 7.48-7.39 (m, 2H), 7.35 (s, 2H), 7.25 (dd, J=8.6, 7.3 Hz, 2H), 7.00-6.87 (m, 1H). ¹³C {¹H} NMR (126 MHz, DMSO) δ 152.6, 139.9, 134.1, 128.7, 121.6, 119.0, 118.1. m.p.<360° C. IR (cm⁻¹) v=3296, 2985, 1628, 1592, 1548, 1506, 1445, 1403, 1295, 1223, 729, 693.

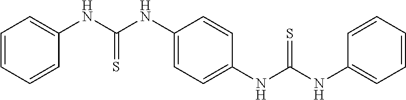 [SMILES: S=C(Nc1ccccc1)Nc1ccc(NC(=S)Nc2ccccc2)cc1]

1-phenyl-3-{4-[(phenylcarbamothioyl)amino]phenyl}thiourea (10): White color solid (514 mg, yield: 91%), R_{f}=0.56 (dichloromethane/MeOH=95:5). ¹H NMR (500 MHz, DMSO) δ 9.77 (d, J=4.7 Hz, 4H), 7.50-7.48 (m, 4H), 7.45 (s, 4H), 7.33 (dd, J=8.5, 7.4 Hz, 2H), 7.15-7.06 (m, 2H) ¹³C {¹H} NMR (126 MHz, DMSO) δ 189.5, 139.5, 135.8, 128.4, 123.8, 123.6, 123.5. m.p.<360° C. IR (cm⁻¹) v=3195, 3200, 1536, 1495, 1337, 1255, 1217, 1021, 746. Physical and spectral data were in agreement with the literature.²³

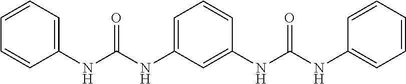 [SMILES: O=C(Nc1ccccc1)Nc1cccc(NC(=O)Nc2ccccc2)c1]

3-phenyl-1-{3-[(phenylcarbamoyl)amino]phenyl}urea (Compound 11):

White color solid (334 mg, yield: 96%). ¹H NMR (500 MHz, DMSO) δ 8.71 (s, 2H), 8.61 (s, 2H), 7.68 (s, 1H), 7.68-7.45 (m, 4H), 7.28 (dd, J=8.5, 7.3 Hz, 4H), 7.17 (dd, J=8.8, 7.3 Hz, 1H), 7.08 (dd, J=7.7, 2.1 Hz, 2H), 6.99-6.96 (m, 2H). ¹³C NMR (126 MHz, DMSO) δ 152.4, 140.1, 139.7, 129.1, 128.8, 121.8, 118.1, 111.6, 107.8. IR (cm⁻¹) v=3297 (b), 1637 (s), 1594 (s) 1554 (s), 1491 (m), 1445 (m), 1297 (m), 1222 (m), 873 (w), 729 (s).

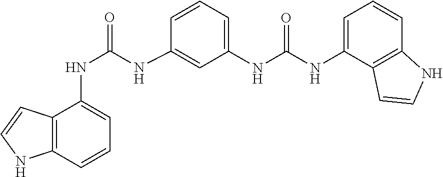 [SMILES: O=C(Nc1cccc(NC(=O)Nc2cccc3[nH]ccc23)c1)Nc1cccc2[nH]ccc12]

3-(1H-indol-4-yl)-1-(3-{[(1H-indol-4-yl)carbamoyl]amino}phenyl)urea (compound 12): Purplish grey color solid (254 mg, yield: 80%). ¹H NMR (500 MHz, DMSO) δ 11.13 (s, 2H), 8.99 (s, 2H), 8.46 (s, 2H), 7.84 (s, 1H), 7.70 (dd, J=7.5, 1.0 Hz, 2H), 7.32 (t, J=2.8 Hz, 2H), 7.22 (dd, J=8.7, 7.3 Hz, 1H), 7.14-7.12 (m, 2H), 7.07-7.06 (m, 2H), 7.03 (t, J=7.8 Hz, 2H), 7.00 (s, 2H). ¹³C NMR (126 MHz, DMSO) δ 152.4, 140.4, 136.4, 131.5, 129.3, 124.0, 121.6, 118.8, 111.2, 107.2, 105.8, 97.7. IR (cm⁻¹) v=3296 (b), 1634 (s), 1548 (s), 1489 (s), 1443 (w), 1407 (w), 1296 (m), 1222 (m), 1189 (m), 1080 (w), 898 (w), 873 (w), 741 (w).

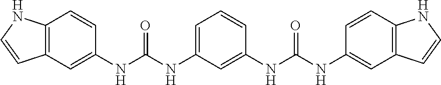 [SMILES: O=C(Nc1cccc(NC(=O)Nc2ccc3[nH]ccc3c2)c1)Nc1ccc2[nH]ccc2c1]

3-(1H-indol-5-yl)-1-(3-{[(1H-indol-5-yl)carbamoyl]amino}phenyl)urea (compound 13):
Pale ash color solid (281 mg, yield: 88%). ¹H NMR (500 MHz, DMSO) δ 10.93 (s, 2H), 8.62 (s, 2H), 8.37 (s, 2H), 7.70-7.68 (m, 3H), 7.32-7.29 (m, 4H), 7.14 (dd, J=8.9, 7.0 Hz, 1H), 7.10-7.07 (m, 4H), 6.36 (s, 2H). ¹³C NMR (126 MHz, DMSO) δ 152.9, 140.6, 132.2, 131.4, 129.0, 127.7, 125.7, 114.7, 111.2, 111.1, 109.8, 107.4, 100.9. IR (cm⁻¹) v=3301 (b), 1634 (s), 1549 (s), 1490 (s), 1405 (w), 1320 (s), 1292 (s), 1221 (s), 1193 (m), 1086 (w), 875 (m).

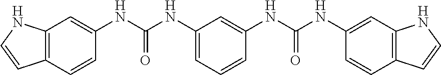 [SMILES: O=C(Nc1cccc(NC(=O)Nc2ccc3cc[nH]c3c2)c1)Nc1ccc2cc[nH]c2c1]

3-(1H-indol-6-yl)-1-(3-{[(1H-indol-6-yl)carbamoyl]amino}phenyl)urea (compound 14):
Off-white color solid (278 mg, yield: 93%). ¹H NMR (500 MHz, DMSO) δ 10.92 (s, 2H), 8.64 (s, 2H), 8.51 (s, 2H), 7.82-7.82 (m, 2H), 7.70 (t, J=2.1 Hz, 1H), 7.42 (d, J=8.4 Hz, 2H), 7.22 (dd, J=3.1, 2.3 Hz, 2H), 7.17 (dd, J=8.8, 7.1 Hz, 1H), 7.10-7.08 (m, 2H), 6.86 (dd, J=8.4, 1.9 Hz, 2H), 6.34 (ddd, J=3.0, 1.9, 0.9 Hz, 2H). ¹³C NMR (126 MHz, DMSO) δ 152.7, 140.4, 136.2, 133.8, 129.0, 124.4, 123.1, 119.9, 111.9, 111.3, 107.6, 101.1, 100.9.

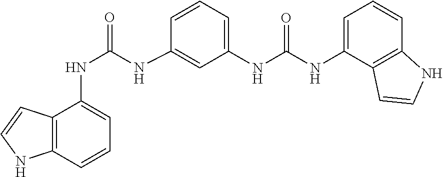 [SMILES: O=C(Nc1cccc(NC(=O)Nc2cccc3[nH]ccc23)c1)Nc1cccc2[nH]ccc12]

3-(1H-indol-4-yl)-1-(3-{[(1H-indol-4-yl)carbamoyl]amino}phenyl)urea (compound 15):

Ash color solid (62 mg, yield: 94%). ¹H NMR (500 MHz, DMSO) δ 10.70 (s, 2H), 8.81 (s, 2H), 8.44 (s, 2H), 7.71 (s, 1H), 7.33 (t, J=2.8 Hz, 2H), 7.22 (d, J=7.9 Hz, 2H), 7.22-7.15 (m, 3H), 7.11 (dd, J=7.6, 1.0 Hz, 2H), 6.95 (t, J=7.7 Hz, 2H), 6.44 (dd, J=3.1, 2.0 Hz, 2H). ¹³C NMR (126 MHz, DMSO) δ 153.0, 140.3, 129.2, 129.0, 128.9, 125.2, 123.7, 119.0, 115.8, 113.6, 111.8, 108.1, 101.5. IR (cm⁻¹) v=3994 (w), 3297 (b), 1642 (s), 1545 (s), 1490 (s), 1435 (w), 1345 (m), 1320 (m), 1293 (m), 1222 (m), 1193 (m), 719 (s).

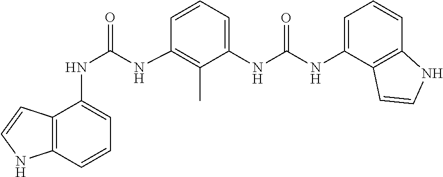 [SMILES: Cc1c(NC(=O)Nc2cccc3[nH]ccc23)cccc1NC(=O)Nc1cccc2[nH]ccc12]

3-(1H-indol-4-yl)-1-(3-{[(1H-indol-4-yl)carbamoyl]amino}-2-methylphenyl)urea (Compound 16):
Pale brown color solid (318 mg, yield: 97%). ¹H NMR (500 MHz, DMSO) δ 11.12 (s, 2H), 8.74 (s, 2H), 8.29 (s, 2H), 7.69-7.67 (m, 2H), 7.50 (d, J=8.1 Hz, 2H), 7.32 (t, J=2.8 Hz, 2H), 7.14 (t, J=8.1 Hz, 1H), 7.07-7.02 (m, 2H), 7.00 (t, J=7.8 Hz, 2H), 6.62 (s, 2H), 2.26 (s, 3H). ¹³C NMR (126 MHz, DMSO) δ 153.0, 137.6, 136.5, 131.7, 125.4, 123.9, 121.6, 120.9, 118.9, 118.1, 107.5, 105.7, 97.9, 12.7. IR (cm⁻¹) v=3297 (b), 1630 (s), 1549 (s), 1474 (s), 1431 (m), 1346 (w), 1243 (m), 1127 (m), 1085 (m), 747 (s).

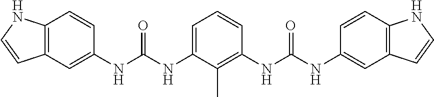 [SMILES: Cc1c(NC(=O)Nc2ccc3[nH]ccc3c2)cccc1NC(=O)Nc1ccc2[nH]ccc2c1]

3-(1H-indol-5-yl)-1-(3-{[(1H-indol-5-yl)carbamoyl]amino}-2-methylphenyl)urea (compound 17):
Light ash color solid (295 mg, yield: 90%). ¹H NMR (500 MHz, DMSO) δ 10.93 (s, 2H), 8.68 (s, 2H), 7.91 (s, 2H), 7.71 (s, 2H), 7.47 (d, J=8.0 Hz, 2H), 7.32-7.29 (m, 4H), 7.11 (d, J=7.69 Hz, 3H), 6.36 (s, 2H), 2.18 (s, 3H). ¹³C NMR (126 MHz, DMSO) δ 153.8, 138.3, 132.6, 132.2, 128.2, 126.1, 125.7, 120.9, 117.9, 115.0, 111.7, 110.1, 101.3, 12.9. IR (cm⁻³) v=3415 (b), 1297 (b), 1634 (s), 1586 (w), 1552 (s), 1474 (m), 1320 (s), 1269 (w), 1227 (m), 880 (m), 798 (m), 757 (m), 726 (m).

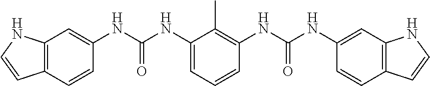 [SMILES: Cc1c(NC(=O)Nc2ccc3cc[nH]c3c2)cccc1NC(=O)Nc1ccc2cc[nH]c2c1]

3-(1H-indol-6-yl)-1-(3-{[(1H-indol-6-yl)carbamoyl]amino}-2-methylphenyl)urea (Compound 18):
Light ash color solid (305 mg, yield: 93%). ¹H NMR (500 MHz, DMSO) δ 10.92 (s, 2H), 8.84 (s, 2H), 7.96 (s, 2H), 7.84 (s, 2H), 7.48 (d, J=8.1 Hz, 2H), 7.42 (d, J=8.4 Hz, 2H), 7.21 (dd, J=3.1, 2.3 Hz, 2H), 7.11 (t, J=8.1 Hz, 1H), 6.87 (dd, J=8.4, 1.9 Hz, 2H), 6.34 (s, 2H), 2.19 (s, 3H). ¹³C NMR (126 MHz, DMSO) δ 153.1, 137.8, 136.3, 134.1, 125.3, 124.3, 122.9, 120.7, 119.9, 117.7, 111.7, 100.9, 100.8, 12.4. IR (cm⁻¹) v=3298 (b), 1633 (m), 1544 (m), 1345 (m), 1320 (m), 1292 (m), 1220 (m), 1087 (m), 798 (m), 758 (m), 716 (m).

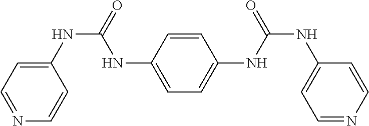 [SMILES: O=C(Nc1ccncc1)Nc1ccc(NC(=O)Nc2ccncc2)cc1]

1,1′-(1,4-phenylene)bis(3-(pyridin-4-yl)urea) (Compound 19)

¹H NMR (500 MHz, DMSO) δ 9.04 (s, 2H), 8.76 (s, 2H), 8.33 (d, J=6.3 Hz, 4H), 7.41 (d, J=6.5 Hz, 4H), 7.38 (s, 4H). ¹³C NMR (126 MHz, DMSO) δ 152.6, 150.6, 147.0, 134.4, 119.8, 112.7.

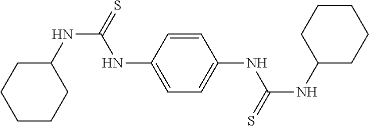 [SMILES: S=C(Nc1ccc(NC(=S)NC2CCCCC2)cc1)NC1CCCCC1]

1,1′-(1,4-phenylene)bis(3-cyclohexylthiourea) (Compound 20)

¹H NMR (500 MHz, DMSO) δ 9.27 (s, 2H), 7.53 (d, J=7.8 Hz, 2H), 7.34 (s, 4H), 4.06 (s, 2H), 1.99-1.79 (m, 4H), 1.66 (dt, J=12.9, 3.7 Hz, 4H), 1.61-1.45 (m, 2H), 1.40-0.98 (m, 10H).

¹³C NMR (126 MHz, DMSO) δ 179.7, 136.0, 123.5, 52.6, 32.4, 25.6 25.0.

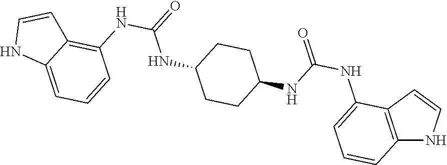 [SMILES: O=C(Nc1cccc2[nH]ccc12)N[C@H]1CC[C@H](NC(=O)Nc2cccc3[nH]ccc23)CC1]

1,1′-((1R,4R)-cyclohexane-1,4-diyl)bis(3-(1H-indol-4-yl)urea) (Compound 21)

¹H NMR (500 MHZ, DMSO) δ 11.02 (s, 1H), 8.10 (s, 1H), 7.63 (dd, J=7.1, 1.5 Hz, 1H), 7.23 (s, 1H), 7.09-6.78 (m, 2H), 6.50 (s, 1H), 6.37 (d, J=7.6 Hz, 1H), 3.51 (s, 1H), 2.07-1.72 (m, 2H), 1.29 (td, J=8.8, 3.2 Hz, 2H). ¹³C NMR (126 MHz, DMSO) δ 155.1, 136.9, 132.9, 124.1, 122.1, 118.9, 107.0, 105.4, 98.2, 48.0, 32.2.

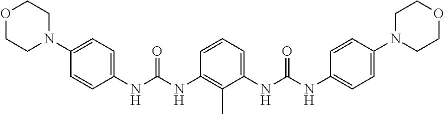 [SMILES: Cc1c(NC(=O)Nc2ccc(N3CCOCC3)cc2)cccc1NC(=O)Nc1ccc(N2CCOCC2)cc1]

1,1′-(2-methyl-1,3-phenylene)bis(3-(4-morpholinophenyl)urea) (Compound 22)

¹H NMR (500 MHZ, DMSO) δ 8.66 (s, 2H), 7.87 (s, 2H), 7.40 (d, J=8.1 Hz, 2H), 7.31 (d, J=8.6 Hz, 4H), 7.06 (t, J=8.1 Hz, 1H), 6.87 (d, J=8.6 Hz, 4H), 3.71 (t, J=4.6 Hz, 8H), 3.00 (t, J=4.7 Hz, 8H), 2.11 (s, 3H). ¹³C NMR (126 MHz, DMSO) δ 153.5, 146.8, 138.2, 132.8, 125.8, 121.2, 119.9, 118.2, 116.4, 66.4, 49.8, 12.9.

Compound 1, a diurea derivative, exhibited the best anti-fibril activity as monitored by ThT fluorescence assay. Symmetric molecules were explored based on previous work on urea (Maity et al. 2022) and amide (Ramirez et al. 2023) compounds using aminoindolyl groups and their anti-oligomeric properties (FIG. 1). The impact of the position of the aminoindolyl group on the kinetics of α-syn fibril formation was assessed using ThT assays by measuring the percentage fluorescence intensities (FI). The FI of the newly synthesized compounds was compared with the control (DMSO), and the reduction of the percentage FI to 15% or less was considered as a cut-off value to move the compound to tier-2 assays. Based on the data obtained for the ten derivatives (i.e., compounds 1-10), 4-aminoindolyl derivatives (compounds 1-2) were greater inhibitors of α-syn fibril formation than the 5-, 6-, and 7-aminoindolyl derivatives (compounds 3-8) (Table 1). In the case of all aminoindolyl groups (compounds 1-8), the 4-aminoindolyl group (compounds 1-2) was the most preferred substituent. The change of the aminoindolyl group for a simple aromatic moiety (compounds 9-10) led to complete loss of the α-syn anti-fibrillary activity.

TABLE 1
Molecular structures of novel diurea- and dithiourea-linked derivatives and their
respective anti-fibrillary activity on α-syn (2 μM final concentration) expressed as maximum ThT
intensity in percentage in which the compounds were tested at 100 μM. Data represents the average
of three replicates with scanning electron microscopy (SEM).

| | % ThT intensity (100 μM) | |
|---|---|---|
| Structure | X = O, Urea | X = S, Thiourea |
| 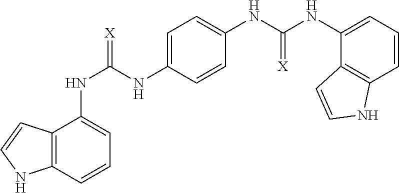 | 1 5.3 ± 0.7 | 2 15.2 ± 0.5 |
| 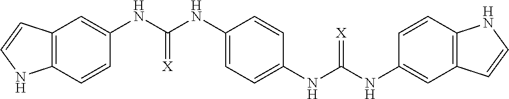 | 3 57.0 ± 12.5 | 4 46.2 ± 2.1 |
| 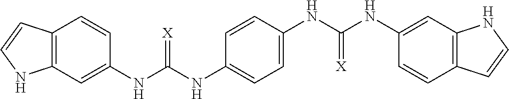 | 5 58.7 ± 7.2 | 6 36.1 ± 5.5 |
| 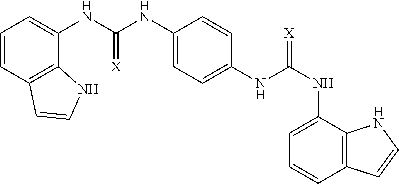 | 7 52.4 ± 15.3 | 8 41.6 ± 1.5 |
| 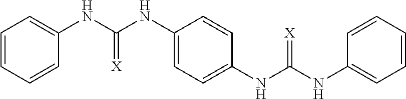 | 9 93.5 ± 16.6 | 10 94.0 ± 3.7 |
| 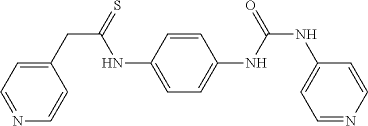 [SMILES: O=C(Nc1ccncc1)Nc1ccc(NC(=S)Cc2ccncc2)cc1] | 19 41.6 ± 3.6 | — |
| 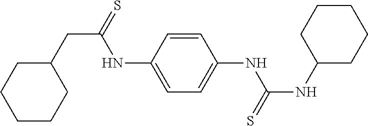 [SMILES: S=C(CC1CCCCC1)Nc1ccc(NC(=S)NC2CCCCC2)cc1] | 20 85% ± <15 | — |
| 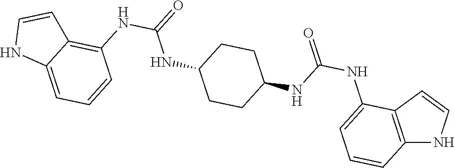 [SMILES: O=C(Nc1cccc2[nH]ccc12)N[C@H]1CC[C@H](NC(=O)Nc2cccc3[nH]ccc23)CC1] | 21 91.6 ± 11.9 | — |
| 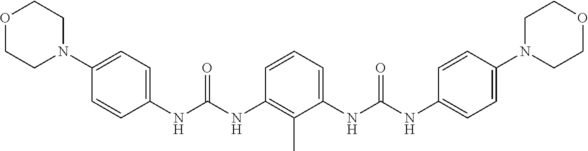 [SMILES: Cc1c(NC(=O)Nc2ccc(N3CCOCC3)cc2)cccc1NC(=O)Nc1ccc(N2CCOCC2)cc1] | 22 93.5 ± 12.3 | — |

Initial molecular docking results showed that analog 1 (−5.55) has a slightly higher binding with a hydrophobic binding pocket on the active site but analog 2 is closer to the hydrophilic binding (−4.63) with three strong hydrogen bonds with three different amino acid residues (Val77, Ala78, and Thr81), which might stabilize the ligand-protein/receptor interaction (FIGS. 1 and 2) (Fu et al., 2018; and Lippert et al., 2009) More stable ligand-receptor complexes can exhibit higher activity in the dynamic biological system (Maltarollo et al., 2012).

Based on the initial ThT and molecular docking results (and later biological activity results), the dose response curve was obtained only for compound 1 (FIG. 3). The resulting Log (agonist) vs. normalized response (variable slope) correlation obtained by Prism provided a Log EC50 of 15.9±2.8. The curve shows a dose-dependent linear response not related to solubility of compounds as the fibril reduction has been confirmed by TEM. In addition, the dose dependency has been demonstrated with cell-based assays at low micromolar concentration.

Additional di-urea compounds were prepared in order to assess the α-syn anti-fibrillar activity of compounds bearing substituents on the aromatic with acute angle. Di-urea compounds were prepared solely due to their anti-fibrillar and anti-inclusion effects. These compounds were tested at the same time as the compounds presented in Table 1.

As expected, the phenyl substituent (compound 11) did not result in an effective anti-fibrillar activity (Table 2). Surprisingly, the 5- and 6-aminoindole-urea substituents at positions 1 and 3 of the aromatic ring (compounds 13-14) exhibited a better α-syn anti-aggregation effect. The presence of a methyl group on the central aromatic ring of the molecule resulted in a greater fluorescence signal, indicative of a weaker α-syn anti-fibrillar effect. These results may suggest that a less linear molecule (i.e., bearing an acute angle between the two aminoindole moieties) might be worthwhile to explore in future studies for the α-syn anti-fibrillary effect.

TABLE 2
Exploration of the orientation of the ureas on the central aromatic right. The anti- fibrillary
activity on α-synuclein (α-syn, 6 μM final concentration) is expressed as maximum thioflavin T
(ThT) intensity in percentage. Compounds were tested with other compounds included in Table 1
at a concentration of 100 μM. Data represent the average of three replicates with

| Px | Py | 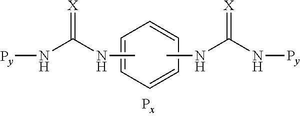 X = O |
|---|---|---|
| 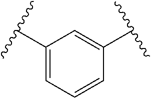 [SMILES: *c1cccc(*)c1] | 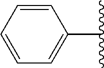 [SMILES: *c1ccccc1] | Compound 11 108.6 ± 4.7 |
| 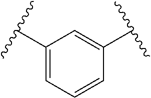 [SMILES: *c1cccc(*)c1] | 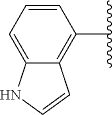 [SMILES: *c1cccc2[nH]ccc12] | Compound 12 8.8 ± 0.2 |
| 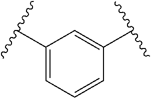 [SMILES: *c1cccc(*)c1] | 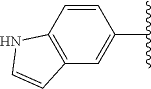 [SMILES: *c1ccc2[nH]ccc2c1] | Compound 13 27.7 ± 2.9 |
| 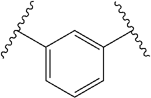 [SMILES: *c1cccc(*)c1] | 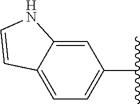 [SMILES: *c1ccc2cc[nH]c2c1] | Compound 14 12.4 ± 1.8 |
| 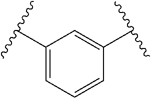 [SMILES: *c1cccc(*)c1] | 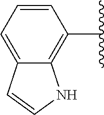 [SMILES: *c1cccc2cc[nH]c12] | Compound 15 28.1 ± 2.1 |
| 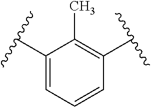 [SMILES: *c1cccc(*)c1C] | 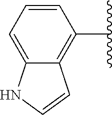 [SMILES: *c1cccc2[nH]ccc12] | Compound 16 23.4 ± 0.2 |
| 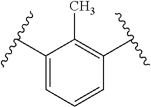 [SMILES: *c1cccc(*)c1C] | 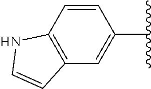 [SMILES: *c1ccc2[nH]ccc2c1] | Compound 17 116.4 ± 8.8 |
| 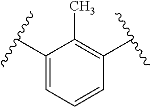 [SMILES: *c1cccc(*)c1C] | 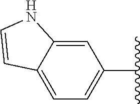 [SMILES: *c1ccc2cc[nH]c2c1] | Compound 18 49.7 ± 5.0 |

Compounds 1, 2, and 8 Exhibited an Anti-Oligomer Activity on α—Syn but not Tau (Isoform 0N4R) and p-Tau (Isoform 1N4R).

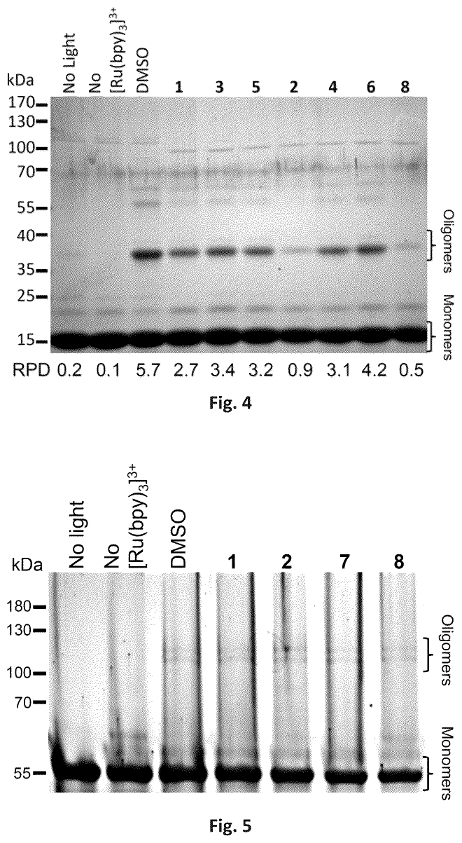

PICUP is a technique utilized to analyze high molecular weight cross-linked protein representative of oligomer formation. FIG. 4 shows α-syn high molecular weight oligomeric species detected around 37 and 55 kDa in the Coomassie blue-stained 16% polyacrylamide gel. DMSO treatment resulted in a high molecular weight band around 37 kDa. Sample not exposed to light or a cross-linking agent demonstrated a prominent monomeric band at 15 kDa. The relative pixel density (RPD) of the DMSO condition was compared with the RPD of each representative treatment. A RPD below 2.85 (i.e., ≥50% reduction of oligomer formation) was considered as the cut off. Compounds 8, 2, and 1 reduced the formation of α-syn oligomers with respective percentage of oligomer reduction of 91.2, 84.2, and 52.6%. Compounds 3, 4, 5, 6 demonstrated mediocre o-syn anti-oligomer effects by PICUP.

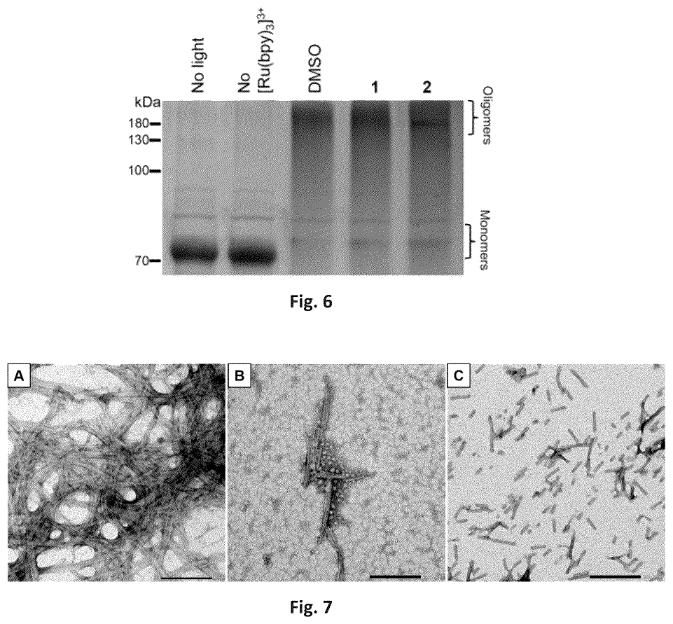

Previous work on dual anti-aggregation effect of compounds on α-syn and tau^{6, 12, 13} spurred the investigation of the anti-oligomer activity on other prone-to-aggregate proteins, such as tau isoform 0N4R (FIG. 5) and p-tau isoform 1N4R (FIG. 6). PICUP experiments were performed with compound 1, 2, 8. Compound 7 was selected because of its weak anti-fibrillar activity on α-syn. Compounds 1-2 and 7-8 are di-substituted with a 4-aminoindolyl and 7-aminoindolyl, respectively. Compounds 1-7 represent the urea counterparts of compounds 2-8, and this justified the inclusion of compound 7 in the PICUP assays. None of these compounds were able to reduce the tau 0N4R oligomer formation by PICUP (FIG. 5). p-Tau isoform 1N4R was challenged with compounds 1 and 2 using PICUP assays. The best anti-fibrillary compounds failed to inhibit p-tau oligomer formation (FIG. 6).

Compounds 1 and 2 induced ultrastructural changes of α-syn fibril.

To confirm the general anti-fibrillar effect of the best inhibitor of fibril formation, i.e., compounds 1 and 2, TEM was utilized as a direct means to detect the ultrastructural changes of α-syn fibrils. Samples were collected at the end of ThT kinetics of aggregation to visualize fibrils and to compare the effects of compound 1 or 2 with DMSO. Compounds and α-syn were tested at 100 and 2 μM, respectively. FIG. 7 shows the photomicrographs acquired at 40K. Application of α-syn resulted in a very dense mat of fibrils at the end of the kinetics of fibril formation (˜22 hours at 37° C.). Both compounds 1 and 2 reduced α-syn fibril formation in comparison with controls (1.5% DMSO). Fibrils were shorter in α-syn samples treated with compounds 1 and 2. However, a few round structures, which could be attributed to oligomers, surrounded a range of shorter fibrils present on the grids of compound 1. This correlates with the PICUP results shown in FIG. 4. Specifically, compound 1 was not able to inhibit fully the formation of oligomer at a molar ratio 1:1 by PICUP.

Cytotoxicity Determination with Neuroblastoma Cells.

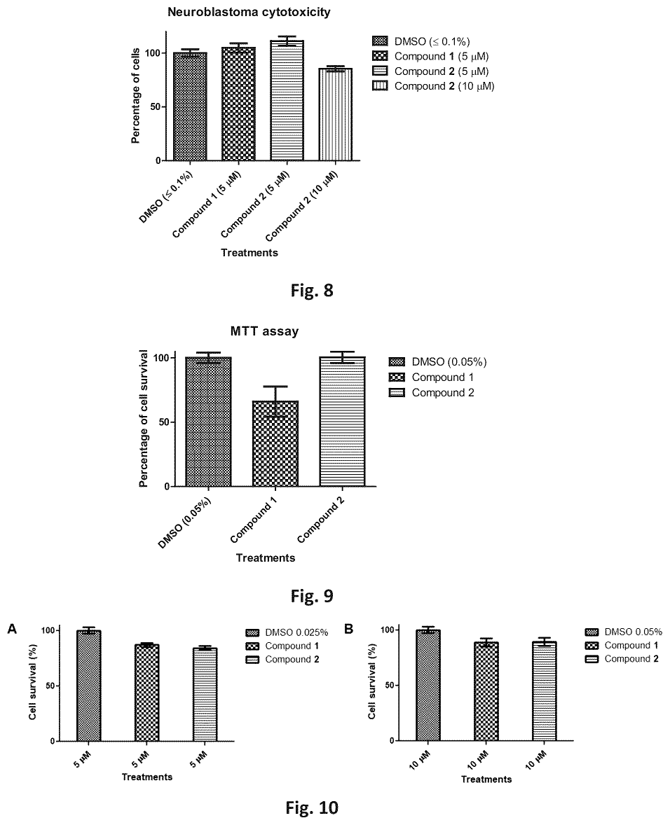

The cytotoxicity of compounds 1 and 2 was assessed using human neuroblastoma cells (SH-SY5Y). Employing the crystal violet staining method, determination of the optical density was used to reflect on the percentage of cells attached to each well (6-well plate format) after treatment with control DMSO (0.14) and compound concentrations at 5 and 10 μM. Treated cells were incubated at 37° C. with 5% CO₂ for four days. The cell-based assay results demonstrated that compounds 1 and 2 were non cytotoxic at the concentration of 5 μM (FIG. 8). Compound 2 was also non cytotoxic at higher concentration, i.e., 10 μM. Differences were not statistically significant at p<0.5 via one-way analysis of variance ANOVA with post-test (Dunnett's multiple comparison test).

MTT (3-(4,5-Dimethylthiazol-2-yl)-2,5-diphenyltetrazolium bromide) is a tetrazolium salt that is commonly used to detect reductive metabolism in cells for viability, proliferation and cytotoxicity assays. MTT was utilized in order to evaluate metabolic cellular toxicity of compounds 1, 2, and DMSO (vehicle) using human neuroblastoma cells (SH-SY5Y). Cells were diluted in cellular media in order to obtain ˜500,000 cells per condition and grown for 8-12 hours prior treatment. Cellular media (see the material and method section below) was then removed. The cells were then treated with compounds 1 or 2 at 20 μM or DMSO control at 0.05% (FIG. 9) diluted in cellular media followed by an additional incubation of 48 hours. Cellular media was removed, and the cells were treated with 50% MTT in cellular media for 2 hours at 37° C. followed by detergent solution. Differences were considered non-significant (at p<0.05) (FIG. 9) based on the one-way analysis of variance (ANOVA) without post-test performed with GraphPad Prism (version 5).

MTT assay was performed once again with compounds 1 and 2 and DMSO control at 5 and 10 μM. Each well contained ˜500,000 cells (6-well plate) prepared 8-12 hours prior to treatment. The cells were treated for 24 hours, and then MTT method was applied to assess cell survival. Compounds 1 and 2 did not induce cytotoxicity at 5 or 10 μM (FIG. 10). No differences were detected at p<0.001 using the one-way analysis of variance (ANOVA) with Dunnett's multiple comparison test.

### Compound 1 Reduced α—Syn Inclusion in Neuroblastoma Cells.

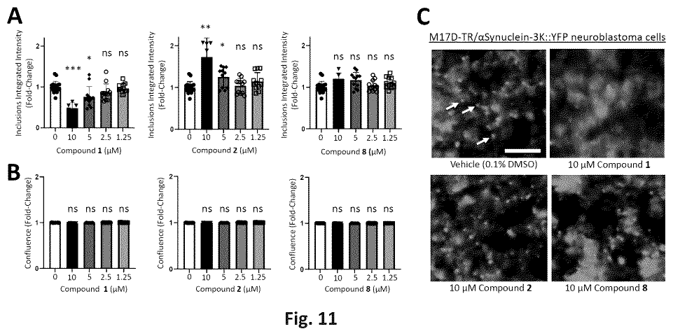

The established dox-inducible neuroblastoma M17D-TR/αS3K::YFP cell-based assay was selected to evaluate the effect of compounds in preventing inclusion formation following induction with Dox (Fortin et al. 2022, Terry-Kantor et al. 2020, and Imberdis et al. 2019) Compound 8 demonstrated a weak effect on fibril formation and inhibitory effect on oligomerization. Treatment of cells with compound 8 resulted in very minimal changes in the numbers of inclusions at various concentrations from 1.25 to 10 μM (FIG. 11). By contrast, compound 1 reduced α-syn inclusion formation at 5 and 10 μM, with the most significant decreases occurring at low micromolar concentration (10 μM). Compound 2, which demonstrated a prominent anti-oligomer effect, increased the inclusions at 5 and 10 μM. Compounds 1, 2, and 8 did not exhibit changes in the cell confluence.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure. The term “or” is used to refer to a nonexclusive “or” unless otherwise indicated.

Unless defined otherwise, all technical and scientific terms used herein have the same meaning as commonly understood to one of ordinary skill in the art. The following terms and phrases shall have the meaning indicated.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value, such as within 10%, within 5%, or within 1% of a stated value or stated limit of a range) provided that one of ordinary skill in the art would consider equivalent to the recited value (e.g., having the same function or result). The term “substantially” can allow for a degree of variability in a value or range, for example, within 90%, within 95%, 99%, 99.5%, 99.9%, 99.99%, or at least about 99.999% or more of a stated value or of a stated limit of a range.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

### REFERENCES

Adeyomoye, O. I., Akintayo, C. O.; Omotuyi, K. P.; Adewumi, A. N. The Biological Roles of Urea. A Review of Preclinical Studies. Indian J Nephrol 2022, 32 (6), 539-545.
Bhat, M. Y.; Mir, I. A; Ul Hussain, M.; Singh, L. R.; Dar, T. A. Urea ameliorates trimethylamine N-oxide-Induced aggregation of intrinsically disordered alpha-casein protein: the other side of the urea-methylamine counteraction. J Biomol Struct Dyn 2023, 41 (8), 3659-3666.
Cardinale, A.; Calabrese, V.; de Iure, A.; Picconi, B. Alpha-Synuclein as a Prominent Actor in the Inflammatory Synaptopathy of Parkinson's Disease. Int J Mol Sci 2021, 22 (12)
Fortin, J. S.; Benoit-Biancamano, M. O.; R, C. G. Discovery of ethyl urea derivatives as inhibitors of islet amyloid polypeptide fibrillization and cytotoxicity. Can J Physiol Pharmacol 2016, 94 (3), 341-346
Fortin, J. S.; Hetak, A. A.; Duggan, K. E.; Burglass, C. M.; Penticoff, H. B.; Schott, H. C., 2nd. Equine pituitary pars intermedia dysfunction: a spontaneous model of synucleinopathy. Sci Rep 2021, 11 (1), 16036.
Fortin, J. S.; Shimanaka, K.; Saraswati, A. P.; Liu, M.; Wang, K. W.; Hagar, H. T.; Maity, S.; Ganegamage, S. K.; Ellsworth, E.; Counts, S. E.; et al. Anti-fibrillization effects of sulfonamide derivatives on alpha-synuclein and hyperphosphorylated tau isoform 1N4R. J Mol Struct 2022, 1267
Fu, Y.; Zhao, J.; Chen, Z. Insights into the Molecular Mechanisms of Protein-Ligand Interactions by Molecular Docking and Molecular Dynamics Simulation: A Case of Oligopeptide Binding Protein. Comput Math Methods Med 2018, 2018, 3502514.
Hara, M.; Minagawa, K.; Imada, Y.; Arakawa, Y. Synthesis of Optically Active Polyguanidines by Polyaddition Reaction of Biscarbodiimides with Chiral Diamines. ACS Omega 2021, 6 (48), 33215-33223.
Imberdis, T., Negri, J.; Ramalingam, N.; Terry-Kantor, E.; Ho, G. P. H.; Fanning, S.; Stirtz, G.; Kim, T. E.; Levy, O. A.; Young-Pearse, T. L.; et al. Cell models of lipid-rich alpha-synuclein aggregation validate known modifiers of alpha-synuclein biology and identify stearoyl-CoA desaturase. Proc Natl Acad Sci USA 2019, 116 (41), 20760-20769.
Jahan, I.; Nayeem, S. M. Effect of Urea, Arginine, and Ethanol Concentration on Aggregation of (179) CVNITV (184) Fragment of Sheep Prion Protein. ACS Omega 2018, 3 (9), 11727-11741.
Kurnik, M.; Sahin, C., Andersen, C. B.; Lorenzen, N., Giehm, L.; Mohammad-Beigi, H.; Jessen, C. M.; Pedersen, J. S.; Christiansen, G.; Petersen, S. V.; et al. Potent alpha-Synuclein Aggregation Inhibitors, Identified by High-Throughput Screening, Mainly Target the Monomeric State. Cell Chem Biol 2018, 25 (11), 1389-1402 e1389.
Lippert, T.; Rarey, M. Fast automated placement of polar hydrogen atoms in protein-ligand complexes. J Cheminform 2009, 1 (1), 13.
Liu, M.; Sui, D.; Dexheimer, T.; Hovde, S., Deng, X.; Wang, K. W.; Lin, H. L.; Chien, H. T.; Kweon, H. K.; Kuo, N. S.; et al. Hyperphosphorylation Renders Tau Prone to Aggregate and to Cause Cell Death. Mol Neurobiol 2020, 57 (11), 4704-4719.
Mahul-Mellier, A. L.; Burtscher, J.; Maharjan, N.; Weerens, L.; Croisier, M., Kuttler, F., Leleu, M.; Knott, G. W.; Lashuel, H. A. The process of Lewy body formation, rather than simply alpha-synuclein fibrillization, is one of the major drivers of neurodegeneration. Proc Natl Acad Sci USA 2020, 117 (9), 4971-4982.
Maity, S., Shimanaka, K., Rivet, L. N., O'Dell, M., Rashid, A. M., Isa, N. B. M., Kepczynski, R. S., Dettmer, U., Borhan, B., Fortin, J. S. In vitro characterization of urea derivatives to inhibit alpha-synuclein early-stage aggregation. Journal of Molecular Structure 2022, 1249.
Maltarollo, V. G; Honorio, K. M. Ligand- and structure-based drug design strategies and PPARdelta/alpha selectivity. Chem Biol Drug Des 2012, 80 (4), 533-544.
Rabilloud, T. Detergents and chaotropes for protein solubilization before two-dimensional electrophoresis. Methods Mol Biol 2009, 528, 259-267
Ramirez, E.; Ganegamage, S. K.; Elbatrawy, A. A.; Alnakhala, H.; Shimanaka, K.; Tripathi, A.; Min, S.; Dettmer, J.-C. R. U.; Fortin, J. S. 5-Nitro-1,2-benzothiazol-3-amine and N-Ethyl-1-[(ethylcarbamoyl) (5-nitro-1,2-benzothiazol-3-yl)amino]formamide Modulate α—Synuclein and Tau Aggregation. ACS Omega 2023.
Ramirez, E., Min, S., Ganegamage, S. K., Shimanaka, K., Sosa, M. G., Dettmer, U., Rochet, J.-C., Fortin, J. S. Discovery of 4-aminoindole carboxamide derivatives to curtail alpha-synuclein and tau isoform 2N4R oligomer formation. Results in Chemistry 2023, 5.
Robustelli, P.; Ibanez-de-Opakua, A.; Campbell-Bezat, C.; Giordanetto, F.; Becker, S.; Zweckstetter, M.; Pan, A. C.; Shaw, D. E. Molecular Basis of Small-Molecule Binding to alpha-Synuclein. J Am Chem Soc 2022, 144 (6), 2501-2510.
Ross, N. T.; Metkar, S. R.; Le, H.; Burbank, J.; Cahill, C.; Germain, A.; MacPherson, L.; Bittker, J.; Palmer, M.; Rogers, J.; et al. Identification of a small molecule that selectively inhibits alpha-synuclein translational expression. In Probe Reports from the NIH Molecular Libraries Program, 2010
Selvaraj, S.; Sundar, S.; Piramanayagam, S. A Computational Approach towards Targeting Parkinson's Disease: The Discovery of Potential Inhibitors against Alpha-Synuclein and Pten-Induced Putative Kinase 1 Protein. Molecular Biology: Open Access 2021, 10 (2), 7.
Shahmoradian, S. H.; Lewis, A. J.; Genoud, C.; Hench, J.; Moors, T. E.; Navarro, P. P.; Castano-Diez, D.; Schweighauser, G.; Graff-Meyer, A.; Goldie, K. N., et al. Lewy pathology in Parkinson's disease consists of crowded organelles and lipid membranes. Nat Neurosci 2019, 22 (7), 1099-1109.
Sui, D.; Xu, X.; Ye, X.; Liu, M.; Mianecki, M.; Rattanasinchai, C.; Buehl, C.; Deng, X.; Kuo, M. H. Protein interaction module-assisted function X (PIMAX) approach to producing challenging proteins including hyperphosphorylated tau and active CDK5/p25 kinase complex. Mol Cell Proteomics 2015, 14 (1), 251-262.
Terry-Kantor, E.; Tripathi, A.; Imberdis, T.; LaVoie, Z. M.; Ho, G. P. H.; Selkoe, D.; Fanning, S.; Ramalingam, N.; Dettmer, U. Rapid Alpha-Synuclein Toxicity in a Neural Cell Model and Its Rescue by a Stearoyl-CoA Desaturase Inhibitor. Int J Mol Sci 2020, 21 (15).
Vittorio, S.; Adornato, I.; Gitto, R.; Pena-Diaz, S.; Ventura, S.; De Luca, L. Rational design of small molecules able to inhibit alpha-synuclein amyloid aggregation for the treatment of Parkinson's disease. J Enzyme Inhib Med Chem 2020, 35 (1), 1727-1735.
Winner, B.; Jappelli, R.; Maji, S. K.; Desplats, P. A.; Boyer, L.; Aigner, S.; Hetzer, C.; Loher, T.; Vilar, M.; Campioni, S.; et al. In vivo demonstration that alpha-synuclein oligomers are toxic. Proc Natl Acad Sci USA 2011, 108 (10), 4194-4199

What is claimed is:

1. A compound of the formula (Ia) or (Ib): 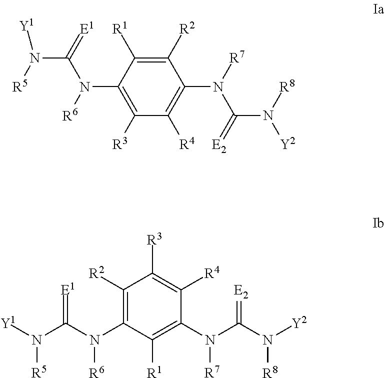
or a pharmaceutically acceptable salt thereof,
wherein:
E¹ and E² are each independently O, S, or NH;
R¹-R⁴ are each independently H, halogen, C₁-C₂₀ alkyl, or C₁-C₂₀ alkoxy;
R¹-R⁴ are each independently H or C₁-C₂₀ alkyl;
Y¹ and Y² are each independently C₃-C₈ cycloalkyl; or 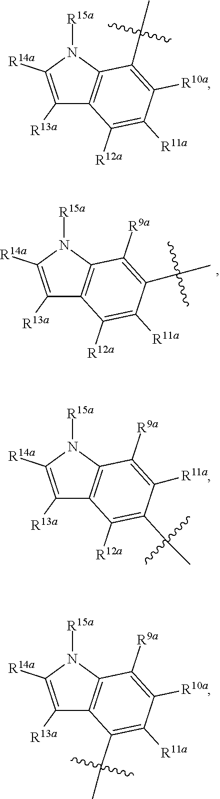 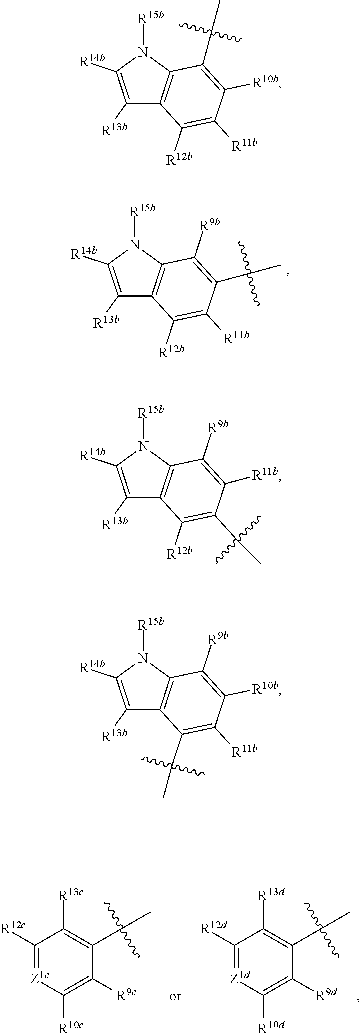
wherein
Z^{1c} is N or C—R^{11c},
Z^{1d} is N or C—R^{11d}
R^{9a}-R^{15a}, R^{9b}-R^{15b}, R^{9c}-R^{15c}, and R^{9d}-R^{15d} are each independently H, halogen, —OH, —NH₂, —NO₂, —CF₃, —CN, —COOH, —N₃, —SO₃H, or —PO₃H₂, C₁-C₂₀ alkyl, C₁-C₂₀ alkoxy, C₂-C₂₀ alkenyl, C₂-C₂₀ alkynyl, C₃-C₁₂ cycloalkyl, C₃-C₁₂ cycloalkenyl, or 3- to 15-membered heterocyclyl group.

2. A compound of the formula (Ic): 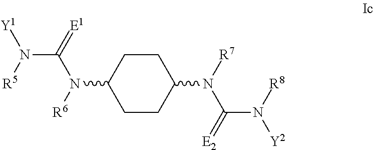
or a pharmaceutically acceptable salt thereof,
wherein:
E¹ and E² are each independently O, S, or NH;
R⁵-R⁸ are each independently H or C₁-C₂₀ alkyl;
Y¹ and Y² are each independently: 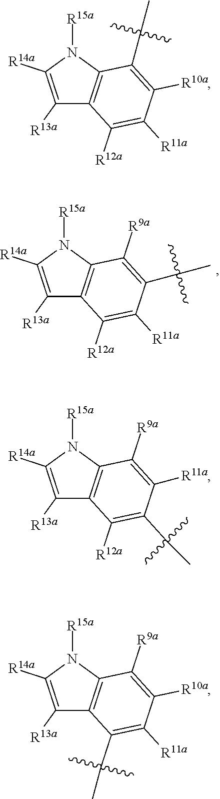 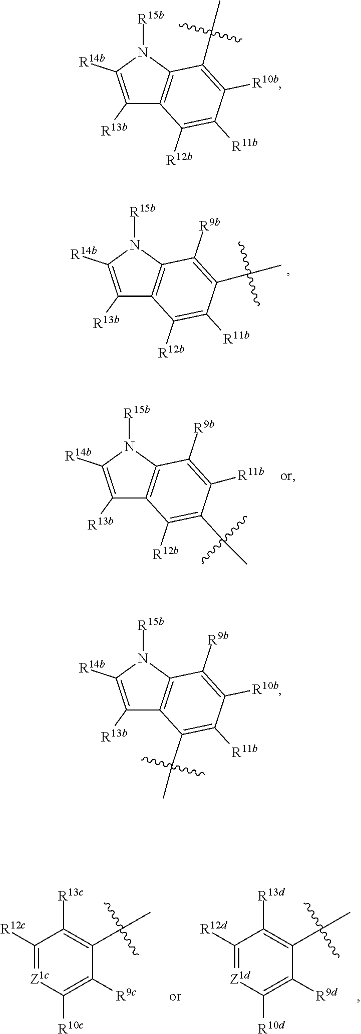
wherein
Z^{1c} is N or C—R^{11c};
Z^{1d} is N or C—R^{11d}
R^{9a}-R^{15a}, R^{9b}-R^{15b}, R^{9c}-R^{15c}, and R^{9d}-R^{15d} are each independently H, halogen, —OH, —NH₂, —NO₂, —CF₃, —CN, —COOH, —N₃, —SO₃H, or —PO₃H₂, C₁-C₂₀ alkyl, C₁-C₂₀ alkoxy, C₂-C₂₀ alkenyl, C₂-C₂₀ alkynyl, C₃-C₁₂ cycloalkyl, C₃-C₁₂ cycloalkenyl, or 3- to 15-membered heterocyclyl group.

3. The compound of claim 2 having the structure

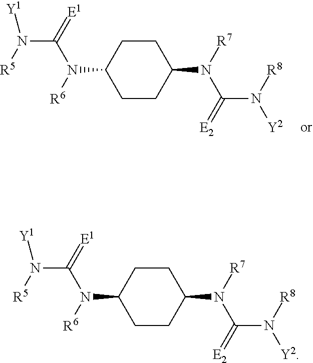

4. The compound of claim 1, wherein E¹ and E² are both O.

5. The compound of claim 1, wherein E¹ and E² are both S.

6. The compound of claim 1, wherein R¹-R⁴ are H.

7. The compound of claim 1, wherein R¹ is alkyl and R²-R⁴ are H.

8. The compound of claim 7, wherein R¹ is methyl.

9. The compound of claim 1, wherein R⁵ and R⁶ are each independently H.

10. The compound of claim 1, wherein R⁷ and R⁸ are each independently H.

11. The compound of claim 1, wherein R⁵-R⁸ are H.

12. The compound of claim 1, wherein Y¹ and Y² are each independently 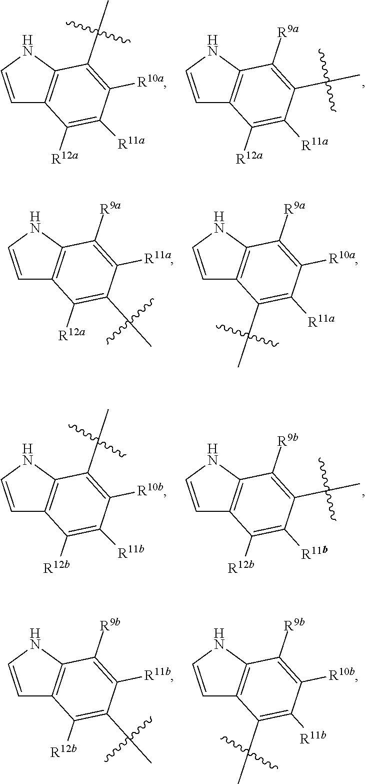
wherein R^{9a}-R^{15a} and R^{9b}-R^{15b} are each independently H, halogen or C₁-C₆ alkyl;
or a pharmaceutically acceptable salt thereof.

13. The compound of claim 12, wherein both Y¹ and Y² are

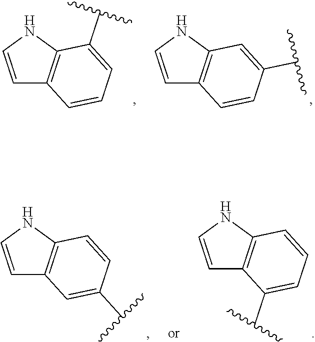 [SMILES: *c1cccc2cc[nH]c12 ; *c1ccc2cc[nH]c2c1 ; *c1ccc2[nH]ccc2c1 ; *c1cccc2[nH]ccc12]

14. The compound of claim 1, wherein Y¹ and Y² are each independently

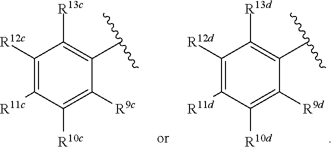

15. The compound of claim 1, wherein R^{11c} and R^{11d} are each independently a 6-membered heterocycle.

16. The compound of claim 1, wherein Y¹ and Y² are each independently

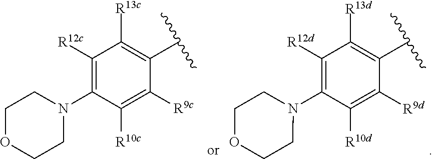

17. The compound of claim 1, wherein Y¹ and Y² are each independently

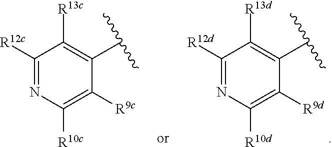

18. A compound of the formula: 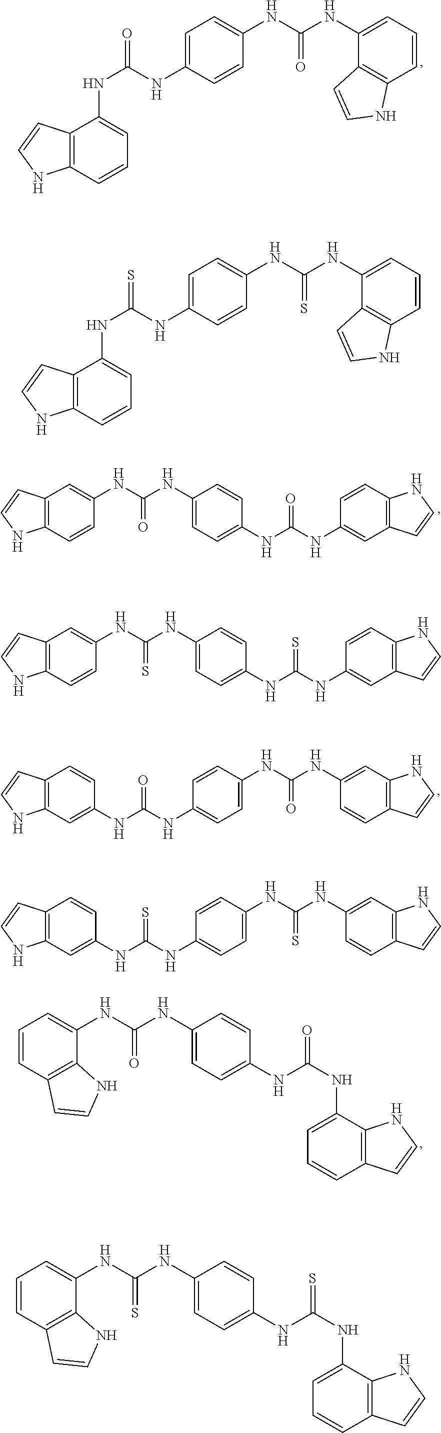 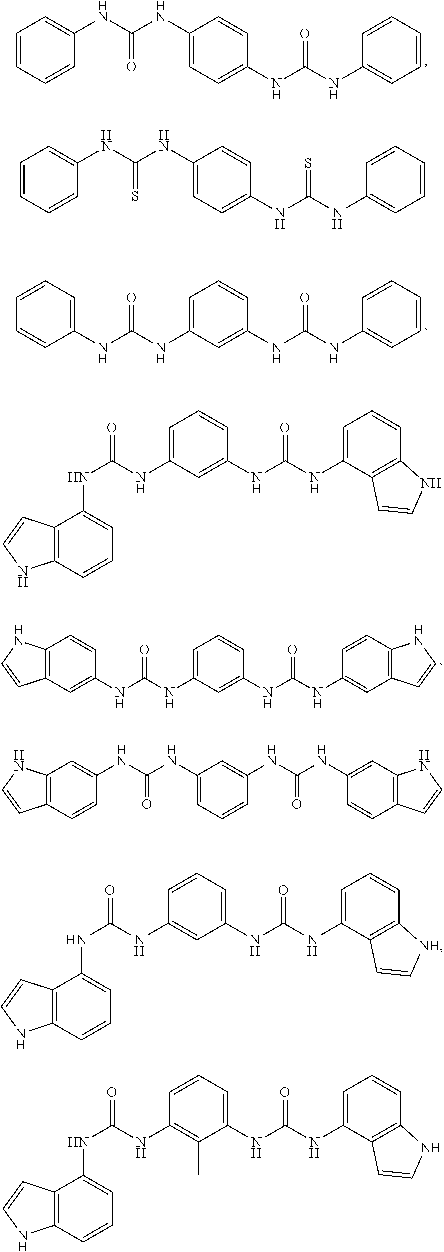 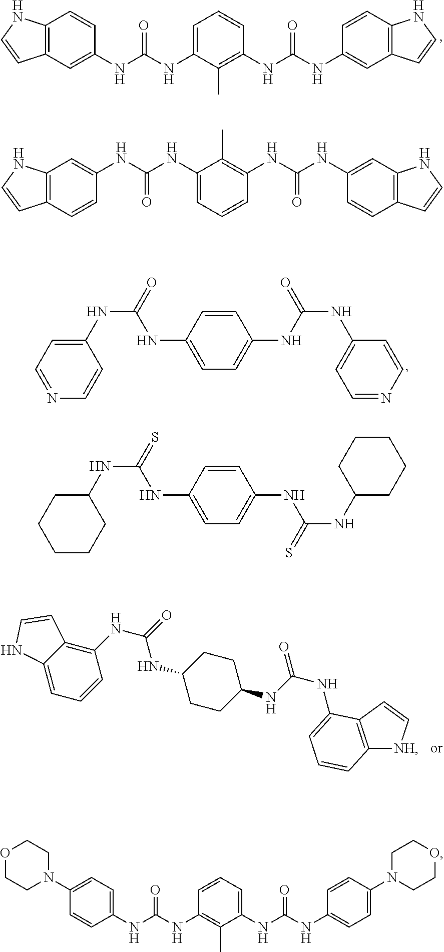
or a pharmaceutically acceptable salt thereof.

19. A pharmaceutical composition comprising the compound of claim 1 and a pharmaceutically acceptable carrier.

20. A method of inhibiting α-synuclein (α-syn) protein aggregation in a subject having, or at risk for, α-syn protein aggregation, comprising administering a compound of claim 1.

21. The method of claim 20, wherein the subject has, or is at risk for, Alzheimer's disease.

22. The method of claim 20, wherein the subject has, or is at risk for, dementia with Lewy bodies (DLB).

23. The method of claim 20, wherein the subject has, or is at risk for, multiple system atrophy (MSA).

24. The method of claim 20, wherein the subject has neuroblastoma and formation of α-syn inclusions is inhibited.
